(19) United States
(12) Patent Application Publication
(10) Pub. No.: US 2026/0272995 A1
(43) Pub. Date: Sep. 17, 2026

(54) Intracellular Delivery of Therapeutic Cargos and Viral RNAS by Engineered Salmonella

(71) Applicant: The Trustees of Columbia University in the City of New York, New York, NY (US)

(72) Inventors: Tal DANINO, Brooklyn, NY (US); Jonathan PABON, New York, NY (US); Zakary SINGER, Los Angeles, CA (US); Charles M. RICE, New York, NY (US)

(21) Appl. No.: 19/408,881

(22) Filed: Dec. 4, 2025

Related U.S. Application Data
(63) Continuation-in-part of application No. PCT/US2024/032961, filed on Jun. 7, 2024.
(60) Provisional application No. 63/471,863, filed on Jun. 8, 2023.
(60) Provisional application No. 63/541,079, filed on Sep. 28, 2023.
(60) Provisional application No. 63/610,152, filed on Dec. 14, 2023.

(51) Int. Cl. A61K 35/74; A61K 38/00; A61P 35/00; C07K 14/005; C07K 14/24; C12N 1/20; C12N 7/00; C12N 9/50; C12R 1/42

(52) U.S. Cl. CPC A61K 35/74; A61P 35/00; C07K 14/005; C07K 14/24; C12N 1/20; C12N 7/00; C12N 9/506; A61K 38/00; C12N 2770/32021; C12N 2770/32032; C12N 2795/00022; C12N 2795/00033; C12R 2001/42; C12Y 304/22044

(57) ABSTRACT

Salmonella typhimurium comprising a first heterologous nucleic acid encoding a first polypeptide that causes bacterial lysis, a second heterologous nucleic acid encoding a second polypeptide that causes vacuolar lysis, and a third heterologous nucleic acid encoding a virus.

### CROSS-REFERENCE TO RELATED APPLICATIONS

The instant application is a bypass continuation-in-part application of International Patent Application No. PCT/US2024/032961 filed 7 Jun. 2024 and claims priority to U.S. Provisional Patent Applications No. 63/471,863 filed on 8 Jun. 2023, 63/541,079 filed on 28 Sep. 2023, and 63/610,152 filed on 14 Dec. 2023, each of which is hereby incorporated by reference in its entirety.

### STATEMENT REGARDING FEDERALLY SPONSORED RESEARCH OR DEVELOPMENT

This invention was made with government support under W81XWH-17-1-0356 awarded by the Medical Research and Development Command, and EB029750 awarded by the National Institutes of Health. The government has certain rights in the invention.

### TECHNICAL FIELD OF THE INVENTION

This disclosure generally relates to the fields of medicine and immunology. More specifically, the disclosure relates to engineered Salmonella typhimurium that produce intact viruses capable of infecting and killing tumor cells, as well as related compositions and methods.

### BACKGROUND OF THE INVENTION

The broad range of applications that employ bacteria and viruses for therapy mirrors the diversity of the microbes themselves. For example, various microbes can target different tissues, microbiomes, and even intra-versus extra-cellular spaces. Examples of bacteria exploiting such characteristics include E. coli that detect irritable bowel disease; S. epidermidis, present in the skin microbiome, made to express tumor antigens; M. pneumoniae releasing an anti-P. aeruginosa bactericide; and L. monocytogenes and Salmonella enterica ser. typhimurium for tumor antigen and apoptotic protein delivery into host-cell cytoplasm.

Perhaps even more diverse are the viral families under investigation for their distinct characteristics. Examples of this assortment include the small ssDNA adeno-associated virus (AAV) whose natural and engineered serotypes provide tissue-specific targeting for monogenic gene therapies; the negative-stranded RNA rabies virus used for retrograde neuronal circuit tracing; the engineered mycobacteriophage to treat lethal, multi-drug resistant mycobacterial infections; and the diminutive but strongly cytopathic plus-stranded RNA viruses, like PVSRIPO, an engineered poliovirus derivative whose receptor binding capacity and host-translational determinants guide tropism for targeting glioblastoma). Together, microbes of broadly distinct evolutionary histories and cellular proclivities have each found utility in exploiting a specific application niche.

Considering the range of benefits that different species can provide, one innovative use of synthetic biology has been the engineering of multiple interacting entities within a consortium to achieve a collective goal. Applications that exploit this type of cooperation include cancer treatment, wound healing, and re-equilibration from gut dysbiosis. In each case, the division of labor between interacting species-even across kingdoms-presents an opportunity to exploit the unique advantages of the constituent elements.

Bacteria and viruses are generally considered separately in approaches to therapeutic delivery. However, combining the two has promising potential: bacteria act as a dynamic envelope for a viral genome, which achieves a nested strategy for viral delivery; potentially provides shielding from innate or adaptive response; confers spatial specificity to the viral infection when made dependent on a bacterially delivered enzyme; and expands on pathogen associated molecular patterns (PAMPs) present as a result of multiple microbial penetrations. Accordingly, the inventions disclosed herein constitute a new complementation system for bacteria-mediated viral delivery and control.

### BRIEF SUMMARY OF THE INVENTION

The present disclosure relates to engineered Salmonella typhimurium that comprise (a) a lysis circuit comprising a first heterologous nucleic acid encoding a first polypeptide that causes bacterial lysis and a second heterologous nucleic acid encoding a second polypeptide that causes vacuolar lysis; and (b)a third heterologous nucleic acid encoding a virus.

In some embodiments, the first polypeptide is lysis protein E from phage (X174 and the second polypeptide is Hemolysin E. In some embodiments, the virus is a picornavirus. In some embodiments, the picornavirus is Seneca A virus.

In some embodiments, the engineered Salmonella typhimurium comprise a fourth heterologous nucleic acid encoding an orthogonal protease, such as the Tobacco Etch Virus protease (TEVp), in order to control the viral life cycle. In some embodiments, the virus is modified to comprise one or more orthogonal cleavage sites. In some embodiments, the third heterologous nucleic acid encoding the virus comprises one or more optimized codons encoding the one or more orthogonal cleavage sites in order to avoid or mitigate single nucleotide polymorphisms and other mutations caused by RNA-dependent RNA polymerases (RdRps). In some embodiments, one or more of the heterologous nucleic acids further comprises an intracellular sensing promoter, such as PsseA or PsseJ.

The present disclosure also relates to methods of treating a tumor in a subject comprising administering a therapeutically effective amount of engineered Salmonella typhimurium described above to the subject. In some embodiments, the engineered Salmonella typhimurium described herein may be administered to a subject or delivered to a tumor in the form of a pharmaceutical composition, which may comprise one or more pharmaceutically acceptable carriers, diluents, or excipients. Intratumoral or intravenous delivery of the engineered Salmonella typhimurium is preferable, but other methods of delivery known in the art are contemplated.

The present disclosure also relates to articles of manufacture useful for treating a tumor. In some embodiments, the articles of manufacture comprise a container comprising engineered Salmonella typhimurium described herein, or pharmaceutical compositions comprising the same, as well as instructional materials for using the same to treat a tumor. In some embodiments, the articles of manufacture are part of a kit that comprises a bacterial culture vessel and/or bacterial cell growth media.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description of the Invention. This summary is not intended to identify key features or essential features of the claimed subject matter, nor is it intended to be used as an aid in determining the scope of the claimed subject matter.

### BRIEF DESCRIPTION OF THE SEVERAL VIEWS OF THE DRAWINGS

FIG. 1 shows how programmed S. typhimurium autonomously lyse in host cytoplasm to launch viral RNA and an essential orthogonal viral protease using the CAPPSID (Coordinated Activity of Prokaryote and Picornavirus for Safe Intracellular Delivery) platform. (1) S. typhimurium carrying synthetic circuits enter mammalian cells via natural effectors encoded on Salmonella Pathogenicity Island 1 (SPI-1). (2) Internalized S. typhimurium within a Salmonella Containing Vacuole (SCV) is designed to sense the intravacuolar space, triggering activation of SPI-2 promoters. (3) Engineered SPI-2 promoters are used to drive the production of viral RNAs (Poliovirus replicon, Senecavirus A (SVA), and TEV protease-dependent SVA), lysing proteins hemolysin-E (HlyE) and E from phage qX174, and TEV protease. (4) Upon successful bacterial and vacuolar lysis, viral RNAs and TEV protease are released into the host cytoplasm. (5) Viral RNAs are translated in the cytoplasm and viral replication is initiated. The maturation of viral particles derived from TEV-dependent SVA requires cleavage by both a virally encoded protease and a bacterially donated TEV protease. The latter cuts at the cognate TEV sequence engineered between the Leader (L) protein and the first protein (VP4) of the structural genes (P1), enabling viral RNA encapsidation. (6) Infectious particles are released into the extracellular space to infect neighboring cells.

FIGS. 2A-2G show how engineered bacteria deliver self-replicating RNA into the cytoplasm of host cells. (A) Intracellular S. typhimurium activates SPI-2 promoters PsseA and PsseJ, to drive mCherry expressed by either promoter. After internalization of bacterium and PsseA-mCherry+T 7pol/PsseJ-HlyE+E activation, intravacuolar S. typhimurium lyse the SCV and themselves, releasing mCherry and T7-driven poliovirus-replicon RNA into the cytoplasm where replication and translation produce reporter GFP. (B) Microscopy images of Hela cells inoculated with S. typhimurium at multiplicity of infection (MOI) 50 carrying Ptac GFP and PsseA-mCherry plasmids. The top panels show constitutive Ptac-GFP and SCV-induced PsseA-mCherry signals at 0 hours post-inoculation (HPI). The bottom panels show respective signals 12 HPI. Scale=500 μm. (C) Quantification of PsseA activation is shown as the mean fluorescent intensities (MFI) of mCherry divided by GFP, where each dot represents a single HeLa cell. At each time point the average over all cells is plotted as a red point on the red line. The initial value is taken 1 hour post-infection (HPI). (D) The top panel shows a circuit diagram of proteins produced by PsseA activation and PT7-driven poliovirus replicon. The bottom panels show smFISH probes binding specifically to the 3′ end of the viral RNA transcribed by bacteria. Micrograph showing DAPI staining of both mammalian and bacterial DNA (left panel), PsseA-mCherry fluorescence from S. typhimurium inside SCVs (center panel) and fluorescent signal from probes specific to viral RNA produced (right). (E) Top panels show representative DIC and mCherry signals of Hela cells inoculated with S. typhimurium at an MOI 50 carrying a PsseA-mCherry plasmid. Bottom panels show Hela cells inoculated with S. typhimurium at MOI 50 carrying the mCherry reporter and lysing proteins. The white arrow indicates a cell with an mCherry signal diffusing through the host cytoplasm. Scale bar is 50 μm. (F) The efficiency of replicon delivery (GFP expressed off poliovirus replicon) and plasmid delivery (GFP expressed off plasmid with pEF-promoter and repeating nuclear localization signals) in S. typhimurium strains that either lyse with HlyE and E proteins, the E protein alone, or do not lyse. (G) Bacteria with lysing circuit and virus-encoding plasmid are used to inoculate Hela cells. DAPI indicates nuclear staining; GFP fluorescence is derived from viral RNA reporter. Red fluorescence signal is an anti-647 secondary antibody against an anti-dsRNA antibody indicating active replication of viral RNA.

FIGS. 3A-3E show how engineered bacteria deliver self-replicating RNA into the cytoplasm of host cells. (A) Quantification of PsseJ activation. The mean fluorescent intensity (MFI) of SPI2-driven mCherry is divided by the mean fluorescent intensity of constitutive pEF-GFP with the mean intensity value plotted as a red curve. Delivery of Poliovirus replicon using different lysing proteins. (B) Hela cells seeded at 20,000/cm² were transfected with replicon with wild-type polymerase, mutated inactive polymerase (GAA), or mock control. HeLa cells were counted 18 hours post-transfection and the MFI of each cell is indicated by a single gray dot cell: From left to right: Blank (Mean±SD) 7±1 (Total cell count) N=22,401, GAA polymerase variant 27±28 N=14666 , GDD polymerase variant 1184±1044 N=14960, P<0.0001, two-sample T-test. (C) Fraction of cells expressing GFP signals indicative of poliovirus replicon replication from S. typhimurium expressing various lysing protein pairs. The lowest rates of delivery are represented in lighter blue shades and higher rates of delivery represented in red shades. (D) H446 Cells seeded at 125,000/cm² were inoculated the next day with S. typhimurium carrying poliovirus replicon with and without the lysing circuits, HlyE+E or only E. Efficiency per well divided by the average delivery efficiency in the HlyE+E group. (E) Time-lapse microscopy images of Hela cells seeded at 25,000/cm² and inoculated with S. typhimurium carrying either lysing circuit HlyE+E (top) or E only (bottom), both with poliovirus replicon. Scale=500 μm.

FIGS. 4A-4E show that lysing S. typhimurium launch full-length oncolytic virus Senecavirus A (SVA) and clears subcutaneous tumors. (A) Schematic of S. typhimurium SPI-2 driven production of viral RNA and lysing proteins allowing the escape of viral RNA from the Salmonella containing vacuole (SCV) and S. typhimurium.Upon release of RNA into the cytoplasm, IRES-mediated translation produces viral proteins necessary for replication of RNA, assembly of the capsid, and packaging of the viral genome into its capsid. (B) H446 cells inoculated with MOI 25 S. typhimurium ΔsifA carrying SPI 2-driven lysis and reporter plasmid, along with SVA-GFP plasmid. The three center panels show time-lapse microscopy at three-time points of spreading SVA-GFP as launched from bacteria. Scale bar=500 μm. The bottom panel shows the time course of SVA-GFP infection throughout the course of the 72-hour acquisition period, projecting time as a color with initial events represented in light blue hues and later events passing through yellow and red hues. (C) The top panel illustrates the experimental outline of in vivo experiment where nude mice were engrafted with H446 cells on bilateral flanks, and right flanks were intratumorally injected with 2.5×10⁶ lysing S. typhimurium carrying SVA-NANOLUC® RNA when tumors reached approximately 150 mm³ 14 days later. The center and bottom panels show IVIS images of nude mice injected with NANOLUC® substrate intratumorally 2 days and 4 days post bacterial inoculation, respectively. (D) The first panel shows growth kinetics of left tumors treated with lysing S. typhimurium with WT-SVA (black), lysing S. typhimurium only (blue), and RPMI (red) over the first two weeks post-inoculation. The mean and standard deviation of the mean are plotted on the graph. The second panel shows growth kinetics of right tumors. The third panel shows survival curves for mice treated with groups annotated in the first panel. Survival benefit observed by log-rank test for each survival curve. (E) The left panel shows the experimental timeline where A/J mice were engrafted with NIE-115 cells. When tumors reached approximately 500 mm³ 14 days later, mice were injected intravenously with 2.5×10⁶ lysing S. typhimurium carrying WT-SVA-NANOLUC® RNA. The middle panel shows the growth trajectories of tumors receiving lysing S. typhimurium with WT-SVA (black), or lysing S. typhimurium with poliovirus replicon (red) (two-way ANOVA with Šídák's post-test, n=4 mice per group). The right panel shows survival curves for groups in Eii (log-rank test, n=4 mice per group)

FIGS. 5A-5H show that lysing S. typhimurium launches full-length oncolytic virus SVA and clears subcutaneous tumors. (A) H446 cells seeded at 125,000/cm² were (top) incubated with SVA-GFP at an MOI 1 for 20 min or preincubated with S. typhimurium ΔsifA at MOI 25 for 1 h prior to SVA-GFP incubation. SVA-GFP-infected cells over 24 hours were projected onto a single frame for each condition. Initial infected cells are represented in light blue with subsequent events depicted as ever darker shades of red. Total events recorded over a 24-hour period. Scale bar=500 μm. The temporal projection plot depicts fraction of cells infected with poliovirus replicon from (left) virus alone (n=2479) and (right) bacteria +virus (n=4108) conditions. T-test derived p=0.42 indicating no significant difference in the fraction of replicon-positive cells under both conditions. Scale bar=500 μm (B) A group of mice that underwent intratumoral injections of right flank tumors with 3×10⁶ S. typhimurium expressing PsseA-driven LuxCDABE when tumors were approximately 150 mm³, as measured by IVIS over time. (C) The radiance of left (red) and right (black) tumors was recorded over the first 4 days and reported as average radiance [p/s/cm²/sr]. (D) Single mouse trajectories of tumor volume over time. Black lines are mice receiving PsseJ-driven intracellular lysing S. typhimurium with WT-SVA-NANOLUC® Blue lines are mice receiving PsseJ-driven intracellular lysing S. typhimurium only. Red lines are mice receiving RPMI. (E) Biodistribution results represented as CFUs/gram of tissue from the spleens, livers, and tumors of mice 7 days after inoculation with the S. typhimurium lysing delivery platform. (F) Mouse weights normalized to initial mouse weights over the course of the first 10 days post-inoculation. (G) The top panel is a schematic of S. typhimurium carrying lysing circuit and WT-SVA-NANOLUC® inoculated onto NIE-115 cells. The bottom panel shows how luminescence was captured by microscopy at 48 and 72 hours post infection after addition of luminescence substrate to identify viral launch and spread. (H) The upper left panel is an illustration summarizing in vivo hind flank engraftment of 5×10⁵ N1E-115 cells on A/J mice and intratumoral injections with lysing S. typhimurium 14 days post-tumor engraftment. The upper right panel shows weight trajectories for mice treated with RPMI (black), lysing Salmonella (red), and lysing Salmonella carrying WT-SVA-NANOLUC® (blue). The lower left panel shows tumor volumes over the first 10 days. The lower right panel shows survival curves for treated mice; p=0.028 for S. typhimurium+WT-SVA relative to RPMI and p=0.038 for lysing Salmonella relative to S. typhimurium+WT-SVA.

FIGS. 6A-6H show engineering control over natural viral protein separation enables dependence on bacterially delivered protease. (A) Schematic of viral protease cleavage at natural cleavage sites (white arrow shade) in between the L-protein and individual structural viral proteins, and their reprogramming as possible sites for exogenous protease cleavage (green arrow shade). (B) H446 cells transfected with PIGGYBAC™ plasmids to express integrated TEV protease (bottom row) and non-transfected H 446 cells (top row) were subsequently transfected with 500 ng of WT-SVA and SVA RNA engineered to express TEV cleavage sequence motifs in place of native cleavage between structural proteins. Images taken 24 hours post-transfection at 10×. Scale bar is 500 μm. (C) Illustration of TEV-mediated cleavage between L-protein and VP 4 in the P1 structural protein domain. TEV site is cleaved by S. typhimurium-provided TEV protease, leaving a 6 amino acid C-terminal addition (ENLYFQ) on L protein, while all other cleavage sites are naturally cleaved by the SVA-proteases. (D) Fraction of H446 cells expressing WT-SVA-GFP (grey), TEVs-SVA-GFP (blue dotted), and TEVs-SVA-GFP+TEVp (solid blue) post-transfection at 12, 24, and 48 hrs. (E) H 446 cells inoculated with S. typhimurium carrying TEVs-SVA-GFP with (top) and without (bottom) TEVp at MOI 50. Time course of bacterially delivered virus with (bottom) and without (top) protease; images shown are a projection of time as a color with initial events represented in light blue hues and later events passing through yellow and red hues over the course of the 72 h experiment (F) Representation of TEV site mutation (ENLYFQ\hat{ }G→ENLYLQ\hat{ }G) addressed by converting TEVs to a mutationally resistant (rTEVs) site (ENLYCQG) above plot for fraction of H446 cells expressing WT-SVA-GFP (grey), rTEVs-SVA-GFP (green dotted), and rTEVs-SVA-GFP +TEVp (solid green) post-transfection at 12, 24, and 48 hrs. (G) Tumors treated with lysing S. typhimurium carrying WT-SVA-NANOLUC® without TEV protease, TEVs-SVA-NANOLUC® with and without TEV protease, and rTEVs-SVA-NANOLUC® with TEV protease were excised 18 hours post-inoculation. Naive H446 cells were inoculated with viral particles from tumor solutions freeze-thawed and centrifuged for viral particle isolation. Each point within a bar represents naive H446 cells infected with viral particles divided by the average of virally infected naive H446 cells from the WT-SVA group. ANOVA evaluation determined no significant difference in means in the WT-SVA-NANOLUC® without TEVp, TEVs-SVA-NANOLUC® with TEV protease, and rTEVs-SVA-NANOLUC® with TEV protease groups. No indication of infectious viral particle production from Naive H 446 cells inoculated with tumors treated with S. typhimurium carrying TEVs-SVA-NANOLUC® without TEV protease, ANOVA test between all groups except TEVs-SVA-without-protease indicated p=0.46. (H) In vivo luminescent signals from nude mice with bilateral hind flank tumors inoculated with S. typhimurium expressing rTEVs-SVA-NANOLUC® with (black) and without (red) TEV protease. Each point on the graph illustrates the mean and standard deviation of the luminescent signal of n=10 tumors at each time point.

FIGS. 7A-7F show engineering control over natural viral protein separation enables dependence on bacterially delivered protease. (A) Western blot showing soluble cellular fraction. Protein Ladder, non-optimized TEV, and optimized TEV for solubility expression shown in blot. 43 kb band indicates TEV-protease fused to carrier protein. Input was normalized by optical density values. (B) (Top) IVIS of TEVp-dependent virus at day 8. (Bottom) Sequencing results of TEVs in corresponding mice. Dots indicate matching expected bases. (C) Transfection of TEVs RNA mutants grown with (bottom panels) and without (top panels) TEVp in H446 cells across three different strains: standard TEV site ENLYFQ\hat{ }G, SVA incorporating ENLYLQ\hat{ }G site, and ENLYCQ\hat{ }G. (D) Tumors treated with lysing S. typhimurium carrying WT-SVA-NANOLUC® without TEVp, TEVs-SVA-NANOLUC® with and without TEVp, and rTEVs-SVA-NANOLUC® with TEVp were excised 18 hours post-inoculation. Ex-vivo luminescent intensities were quantified with each point representing an individual tumor luminescence divided by the luminescent intensity of the WT-SVA-NANOLUC® group. ANOVA evaluation determined no significant difference in the means of groups evaluated. (E) Bacteria delivering SVA genome without TEVp dependence (WT-SVA-NANOLUC®) (left), TEVs-SVA-NANOLUC®+TEVp (middle), and rTEVs-SVA-NANOLUC®+TEVp (right) were injected into subcutaneous hind-flank H446 tumors in mice. 18 h later, tumors were isolated, homogenized, freeze-thawed, and clarified to establish resulting viral stocks. 200 μL of stock was inoculated on naive H 446 cells and imaged 12 h and 24 h post-inoculation, plotted as the fold change in the fraction of infected cells observed between the two time points. 5 mice were included per condition. (F) Single mouse trajectories of tumor radiance over time. Black lines are mice receiving bacterially-delivered TEVp-dependent virus along with TEVp. Red lines are mice receiving bacterially-delivered TEVp-dependent virus without TEVp.

FIG. 8 provides schematic representation and time-lapse phase and fluorescence microscopy of the bacterial delivery of recombinant coxsackievirus-encoded GFP constructs into 4T1 cells. S. typhimurium is engineered with SPI-2-regulated lysis and T7-driven expression plasmids encoding either Senecavirus A-GFP or Coxsackievirus B3-GFP genomes. Exchange of the viral cassette between constructs is indicated. Below the schematic, time-lapse phase and fluorescence microscopy of 4T1 cells infected with S. typhimurium (MOI=50) carrying the CVB3-GFP plasmid, imaged at 24, 48, and 72 hours post-infection (HPI). Progressive GFP expression and cytopathic spread reflect active viral replication and release. Scale bar, 1000 μm.

FIG. 9 shows functional assays of engineered TEV-Senecavirus constructs in 446 cells inoculated with S. typhimurium carrying constructs (MOI=50). Schematic illustrations of constructs encoding TEV-protease-responsive Senecavirus genomes expressing NanoLuc or NanoLuc-GM-CSF fusions, evaluated with and without TEV co-expression. 446 cells were infected with S. typhimurium carrying the indicated reporter plasmids, and lysates or supernatants were collected 48 hr post-infection. Luminescence quantification of NanoLuc activity at 48 hours was measured and GM-CSF ELISA of cell pellets (P) and supernatants(S) at 24 and 48 hours wa also measured, demonstrating TEV-dependent release of GM-CSF into the supernatant. Data represent mean±SD of biological replicates

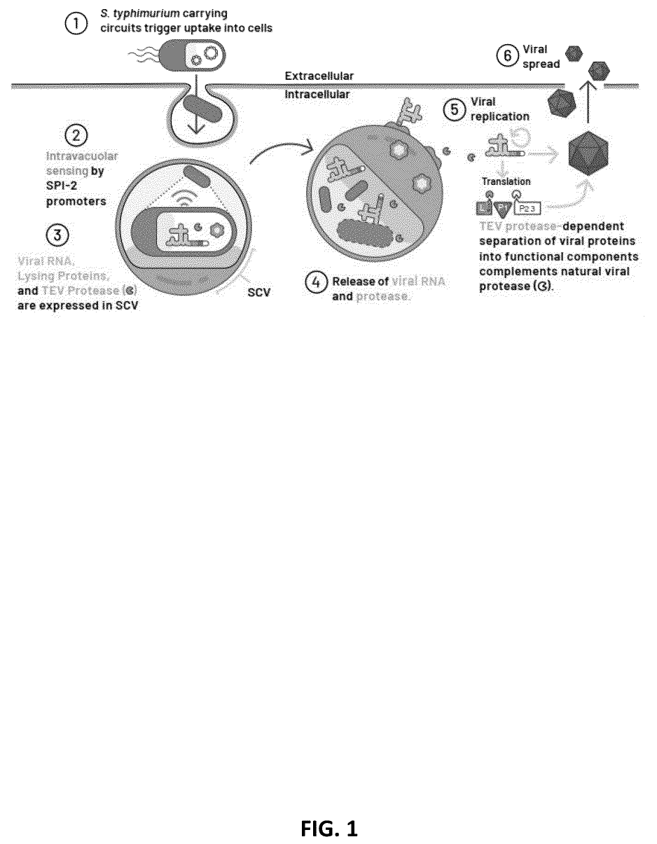

### DETAILED DESCRIPTION OF THE INVENTION

While the present invention may be embodied in many different forms, disclosed herein are specific illustrative embodiments thereof that exemplify the principles of the invention. It should be emphasized that the present invention is not limited to the specific embodiments illustrated. Moreover, any section headings used herein are for organizational purposes only and are not to be construed as limiting the subject matter described.

Unless otherwise defined herein, scientific, and technical terms used in connection with the present invention shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like.

In addition, ranges provided in the specification and appended claims include both end points and all points between the end points. Therefore, a range of 1.0 to 2.0 includes 1.0, 2.0, and all points between 1.0 and 2.0.

The term “about” as used herein when referring to a measurable value such as an amount, a temporal duration, and the like, is meant to encompass variations of .+−.20%, .+−.10%, .+−.5%,. +−.1%, or .+−.0.1% from the specified value, as such variations are appropriate to perform the disclosed methods.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or lists of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of “consisting essentially of,” when used in the claims, shall have its ordinary meaning as used in the field of patent law.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of shall be closed or semi-closed transitional phrases, respectively.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art.

### Engineered Salmonella typhimurium

The inventions described herein generally relate to engineered variants of Salmonella typhimurium comprising (a) a lysis circuit comprising a first heterologous nucleic acid encoding a first polypeptide that causes bacterial lysis and a second heterologous nucleic acid encoding a second polypeptide that causes vacuolar lysis; and (b) a third heterologous nucleic acid encoding a virus.

The term “heterologous nucleic acid sequence” refers to a nucleic acid derived from a different organism that encodes for a protein and which has been recombinantly introduced into a cell. In some embodiments, the heterologous nucleic acid sequence is introduced into a cell by transformation in order to produce a recombinant bacterial cell. Methods for creating recombinant bacterial cells are well known to those of skill in the art. Such methods include, but are not limited to, heat shock transformation, electroporation, liposome-mediated transfection, DEAE-Dextran-mediated transfection, or calcium phosphate transfection. Multiple copies of a heterologous nucleic acid sequence (e.g., between 2 and 10,000 copies) may be introduced into the cell. A heterologous nucleic acid may also encode several proteins or entire genome (e.g., a viral genome).

In some embodiments, heterologous nucleic acid sequences are provided in a plasmid. In some embodiments, the plasmid further comprises at least one promoter that is in operable linkage with one or more of the heterologous nucleic acid sequences. As used herein, the term “promoter” means at least a first nucleic acid sequence that regulates or mediates transcription of a second nucleic acid sequence through some manner of operable linkage. A promoter may comprise nucleic acid sequences near the start site of transcription that are required for proper function of the promoter. As an example, a TATA element for a promoter of polymerase II type. Promoters can include distal enhancer or repressor elements that may lie in positions from about 1 to about 500 base pairs, from about 1 to about 1,000 base pairs, from 1 to about 5,000 base pairs, or from about 1 to about 10,000 base pairs or more from the initiation site.

An “operable linkage” refers to an operative connection between nucleic acid sequences, such as for example between a control sequence (e.g., a promoter) and another nucleic acid sequence that codes for a protein, i.e., a coding sequence. If a promoter can regulate transcription of the nucleic acid sequence, then it is in operable linkage with the nucleic acid sequence. In some embodiments, engineered variants of Salmonella typhimurium disclosed herein comprise a heterologous nucleic acid sequence encoding T7 RNA polymerase and a T7 promoter that is in operable linkage with the lysis circuit comprising the first heterologous nucleic acid encoding the first polypeptide that causes bacterial lysis, the second heterologous nucleic acid encoding the second polypeptide that causes vacuolar lysis, and the third heterologous nucleic acid encoding the virus. In other embodiments, engineered variants of Salmonella typhimurium disclosed herein comprise a heterologous nucleic acid sequence encoding sp6 RNA polymerase and a sp6 promoter that is in operable linkage with the lysis circuit comprising the first heterologous nucleic acid encoding the first polypeptide that causes bacterial lysis, the second heterologous nucleic acid encoding the second polypeptide that causes vacuolar lysis, and the third heterologous nucleic acid encoding the virus.

S. typhimurium is a naturally facultative intracellular bacterium. S. typhimurium achieves invasion into host cells via macropinocytosis, and then survives within the Salmonella Containing Vacuole (SCV) by expressing a battery of genes encoded on Salmonella Pathogenicity Islands 1 and 2 (SPI-1 and SPI-2), respectively. SPI-2 promoters belonging to the sseA and sseJ genes, which are upregulated in the SCV, are utilized to drive viral RNA transcription in situ. In some embodiments, the virus is Senecavirus A (SVA), a virus known to infect H446 small cell lung cancer cells and other cells with neuroendocrine origins.

Once transcribed, the viral genome must exit the bacterium and translocate through the SCV into the cytoplasm of the mammalian host in order to replicate. To optimize efficiency of this 2-step translocation, two distinct bacterial lytic proteins are utilized to form a lysis circuit: Lysis protein E from phage qX174 that disrupts bacterial membranes, which allows viral RNA to exit the lysed bacterium, and Hemolysin E (HlyE), which forms pores in the SCV, allowing the viral RNA to enter the host cytosol. In some embodiments, these genes are expressed under the control of an intracellular sensing promoter, e g., PsseJ.

Senecavirus A is picornavirus, which is known to infect swine and cause vesicular disease. In the life cycle of picornaviruses, all proteins are translated first as one large open reading frame. This polyprotein must be then cleaved into individual constituents entirely by virally encoded proteases. Shifting a cleavage event to an orthogonal protease expressed by bacteria would enable a control over the viral life cycle and such a protease might be delivered simultaneously by lysing bacteria. In some embodiments, the orthogonal protease is Tobacco Etch Virus protease (TEVp). However, shifting a cleavage event requires that the heterologous nucleic acid (or acids) encoding the viral genome be modified to alter natural cleavage sites so that the resultant protein can be cleaved by the orthogonal protease, i.e., the virus must comprise one or more orthogonal cleavage sites. In some embodiments, the codons that encode the protease cleavage site may be optimized, i.e., intentionally designed with sequences that avoid or mitigate single-nucleotide polymorphisms (SNPs) and other mutations that can be caused by, e.g., RNA-dependent RNA polymerases (RdRps) or other sources.

Some aspects of the inventions described herein implicitly relate to culturing the engineered bacterial cells described herein. In some embodiments, a culture comprises the engineered bacterial cells and a medium, for example, a liquid medium, which may also comprise: a carbon source, for example, a carbohydrate source, or an organic acid or salt thereof; a buffer establishing conditions of salinity, osmolarity, and pH, that are amenable to survival and growth; additives such as amino acids, albumin, growth factors, enzyme inhibitors (for example protease inhibitors), fatty acids, lipids, hormones (e.g., dexamethasone and gibberellic acid), trace elements, inorganic compounds (e.g., reducing agents, such as manganese), redox-regulators (e.g., antioxidants), stabilizing agents (e.g., dimethyl sulfoxide), polyethylene glycol, polyvinylpyrrolidone (PVP), gelatin, antibiotics (e.g., Brefeldin A), salts (e.g., NaCl), chelating agents (e.g., EDTA, EGTA), and enzymes (e.g., cellulase, dispase, hyaluronidase, or DNase). In some embodiments, the culture may comprise an agent that induces or inhibits transcription of one or more genes in operable linkage with an inducible promoter, for example doxicycline, tetracycline, tamoxifen, IPTG, hormones, or metal ions. Methods and culture conditions for the generation of microbial cultures are well known to those of skill in the art.

### Therapeutic Methods and Compositions

The inventions described herein also encompass methods of treating a subject, comprising administering the engineered Salmonella typhimurium described herein to a subject in need of treatment. As used interchangeably herein, “treatment” or “treating” or “treat” refers to all processes wherein there may be a slowing, interrupting, arresting, controlling, stopping, alleviating, or ameliorating symptoms or complications, or reversing of the progression of cancer, but does not necessarily indicate a total elimination of all disease or all symptoms. Non-limiting indicia of successful treatment include reducing the rate of growth of a tumor, reducing the size of a tumor, or preventing the metastases of a tumor.

Engineered Salmonella typhimurium described herein are preferably administered in one or more therapeutically effective doses. As used herein the terms “therapeutically effective dose” means the number of cells per dose administered to a subject in need thereof that is sufficient to produce one or more indicia of success (e.g., reducing the rate of growth of a tumor, reducing the size of a tumor, or preventing the metastases of a tumor). In some embodiments, a therapeutically effective dose can be at least about 1×10⁴ cells, at least about 1×10⁵ cells, at least about 1×10⁶ cells, at least about 1×10⁷ cells, at least about 1×10⁸ cells, at least about 1x109 cells, or at least about 1×10¹⁰ cells. Engineered Salmonella typhimurium described herein are preferably administered to a subject intratumorally, but other methods of delivery that are known in the art are contemplated.

In some embodiments, engineered Salmonella typhimurium may be delivered to a subject in the form of a pharmaceutical composition, which may comprise one or more pharmaceutically acceptable carriers, diluents, or excipients. Pharmaceutical compositions may be formulated as desired using art recognized techniques. Various pharmaceutically acceptable carriers, which include vehicles, adjuvants, and diluents, are readily available from numerous commercial sources. Moreover, an assortment of pharmaceutically acceptable auxiliary substances, such as pH adjusting and buffering agents, tonicity adjusting agents, stabilizers, wetting agents, and the like, are also available. Certain non-limiting exemplary carriers include saline, buffered saline, dextrose, water, glycerol, ethanol, and combinations thereof. Pharmaceutical compositions may be frozen and thawed prior to administration or may be reconstituted in WFI with or without additional additives (e.g., albumin, dimethyl sulfoxide). Engineered bacterial cells described herein are preferably formulated for oral, intravenous or intratumoral administration, but other routes of administration known in the art may be utilized.

Particular dosage regimens, i.e., dose, timing, and repetition, will depend on the particular subject being treated and that subject's medical history. Empirical considerations such as pharmacokinetics will contribute to the determination of the dosage. Frequency of administration may be determined and adjusted over the course of therapy and is based on reducing the number of tumor cells or tumor mass, maintaining the reduction of such tumor cells or tumor mass, reducing the proliferation of tumor cells or an increase in tumor mass, or delaying the development of metastasis. A therapeutically effective dose may depend on the mass of the subject being treated, his or her physical condition, the extensiveness of the condition to be treated, and the age of the subject being treated.

### Articles of Manufacture

The inventions disclosed herein also encompass articles of manufacture useful for treating a tumor comprising a container comprising engineered Salmonella typhimurium described herein, or a pharmaceutical composition comprising the same, as well as instructional materials for using the same to treat the tumor. In some embodiments, the articles of manufacture are part of a kit that comprises a bacterial culture vessel and/or bacterial cell growth media.

### EXAMPLES

The following examples have been included to illustrate aspects of the inventions disclosed herein. In light of the present disclosure and the general level of skill in the art, those of skill appreciate that the following examples are intended to be exemplary only and that numerous changes, modifications, and alterations may be employed without departing from the scope of the disclosure.

### Example 1

### Materials and Methods

### Plasmids

Synthetic circuit plasmids were deposited on ADDGENE®. Unless specified elsewhere, all constructs produced were initially synthesized and assembled or modified using NEB HiFi Assembly. All viral vectors were cloned into the same p15A/amp backbone flanked by a 5′ T7 promoter, and 3′ polyA tail, HDV ribozyme, and T7-terminator plasmids were constructed using HiFi Assembly (NEB NEBuilder HiFi DNA Assembly Master Mix) and transformed into NEB100. The lysis circuits were constructed by synthesizing gBlocks from IDT, and cloned into plasmids with sc101 origins of replication. Viral genome-containing plasmids with p15A origins. To verify assemblies whole plasmid sequencing was performed using service provided by Plasmidsaurus.

### Bacterial Cell Growth

All bacteria were grown in LB Lennox broth. For LH1301, cultures were supplemented with ampicillin (100 μg/mL) and spectinomycin (200 μg/mL) for viral-encoding plasmids and lysis circuit plasmids, respectively. Plasmids were cloned into NEB10β and maintained at 100 μg/mL ampicillin or 100 μg/mL spectinomycin. To prepare strains of LH1301 containing these plasmids, the cells were electroporated, recovered, and plated on LB agar. All liquid cultures were grown overnight in a 30° C. incubator with shaking.

Bacterial genome engineering The sifA gene was deleted using the λ-Red recombination system LinearDNA containing CmR flanked by FRT sites were PCR amplified using pkD3 plasmid and electroporated into LH1301 carrying pKD46 plasmid. Chromosomal deletions were verified by PCR and Sanger sequencing.

### Mammalian cell growth and manipulation

HeLa (CRL-1958), 4T1 (CRL-2539), B16 (CRL-6475), HCT116 (CRL-247), and H446 (HTB-171) were acquired from ATCC, and MC-38 from Kerafast (ENH204-FP). All cell lines were cultured in RPMI supplemented with 10% Fetal Bovine Serum (FBS, Gibco) and 1X MEM Non-Essential Amino Acids (NEAA) in a 37° C. tissue culture incubator with 5% CO₂. To stably and constitutively express TEVp in H 446, cells were selected and regularly grown with 0.25 μg/mL puromycin after transfection with PB-PEF-Puro-F2A-TEVp and PIGGYBAC™ super-transposase.

### S. typhimurium Invasion Assay

Cultures of S. typhimurium were grown to stationary phase overnight and diluted 1:100 the following morning into LB Miller supplemented with antibiotics. Cultures were grown at 37° C. to OD₆₀₀ 0.4-0.6, when they were spun down and resuspended in 1 ml RPMI+1% FBS. Bacteria were added at an MOI 50 into 24 well plates of mammalian cells split 24 h prior, and spun down in the plate at 200× G for 5 minutes. Plates were then incubated at 37° C., 5% CO₂ for 30 minutes. Then, the wells were thoroughly washed with RPMI to remove bacteria, incubated for another 30 minutes at 100 μg/mL Gentamicin in RPMI+10% FBS to kill residual extracellular bacteria, and then replaced with 25 μg/mL Gentamicin in RPMI+10% FBS for the duration of the experiment. To stain nuclei, NUCBLUE™ (Invitrogen R37605) was added at 10-20 μL per ml of media.

### Viral Transfection and Production

To produce virus, H446 cells were transfected using LIPOFECTAMINETM MESSENGERMAX™ at 1μL reagent per 500 ng RNA per 24 well plate, and scaled up as needed. 48 hours post transfection, cells were harvested, freeze-thawed three times to disrupt membranes and release viral particles, and clarified by centrifugation at 16,000× g for 10 minutes. The virus was titered on H446 by using serial dilutions and identifying the concentration of virus infecting roughly 50% of cells in a well, plated at 100,000/cm² and left for 24 hours prior to imaging luminescence or GFP reporter signal, defined as MOI.

Animal tumor models All animal experiments were approved by the Institutional Animal Care and Use Committee (Columbia University, protocol AC-AABQ 5551). Female nude mice aged 6-8 weeks from Charles River were grafted with bilateral subcutaneous hind flank tumors of 5×10⁶ H446 cells in 50% reduced growth factor Matrigel (Corning). Tumors were grown until reaching ~150 mm³ over ~2.5 weeks. Then, 2.5×10⁶ bacteria in 25 μL RPMI (without phenol red) were injected intratumorally. Mice were then injected sub-tumorally with 25 μL NANO-GLO® In vivo Substrate (Promega CS320501) at 8.8 nM for IVIS imaging. Tumor volume was quantified using digital calipers to measure the length and width of each tumor (V=0.5*L×W²). The protocol required animals to be euthanized when tumor burden reaches 2 cm in diameter or under veterinary staff recommendation. Mice were randomized into various groups in a blinded manner.

### Biodistribution

After time points following bacterial injections indicated in Fig. legends, mice were euthanized to collect the tumors, spleen, and liver. Tissues were weighed and homogenized using a GENTLEMACS® tissue dissociator (Miltenyi Biotec; C-tubes). These homogenates were then 10- fold serially diluted and plated on LB agar with chloramphenicol and grown overnight at 37° C. Colonies were counted and computed as cfu per gram of tissue.

### Ex Vivo Tumor Luminescence

Tumors were extracted, weighed, and homogenized using the GENTLEMACS® tissue dissociator in 5 ml RPMI+1% FBS. To measure luminescence, samples were serially diluted 10-fold over 4 orders of magnitude, in replicate, and assayed by plate reader (Tecan Infinite 200 Pro using the i-control software version 2.0.10.0) after adding NANO-GLO® In vivo Substrate.

### Ex Vivo Tumor Titering

Tumors were extracted, weighed, and homogenized using the GENTLEMACS® tissue dissociator in 1 mL RPMI+1% FBS with anti-anti (Gibco). Homogenate was freeze-thawed three times and clarified by centrifugation at 16,000× g for 10 minutes. Three 10-fold serial dilutions of this homogenized preparation were inoculated on naive H446 cells for 1 hr, and then replaced with fresh RPMI+10% FBS. 12 h later, cells were dissociated by pipetting and imaged to count the number of luminescent cells after adding NANO-GLOR In vivo Substrate.

### Statistical Analysis

Statistical tests and corresponding plots were performed and corresponding plots created using either GRAPHPAD PRISM® 9.0 or MATLAB® 2023 A. The details of the statistical tests were indicated in the respective Fig. legends. When data were approximately normally distributed, values were compared using a Student's t-test with Bonferroni correction for multiple comparisons. Mice were randomized into different groups before the experiments.

### ImmunoFISH Sample Preparation and Staining

Cells were fixed in 4% formaldehyde for 5 minutes at room temperature, washed twice in PBS, and placed in 70% ethanol overnight at −20° C. For staining dsRNA, the next morning, cells were washed twice with PBS; permeabilized in 0.1% TRITON-X® for 5 minutes min; washed twice in PBS; blocked in 10% Normal Goat Serum (Thermo 50062Z); washed twice with PBS; incubated with J2 primary-antibody (Scicons 10010200) at 0.5 μg/mL in 10% Normal Goat Serum for 2 hours; washed twice with PBS; incubated with goat anti-Mouse Alexa Fluor 647 secondary antibody (Invitrogen A-21235) at (room temperature) RT for 30 minutes; washed twice in PBS and incubated with 10% Normal Goat Serum for an additional 10 minutes at RT, and then imaged in PBS.

For smFISH staining, probes were designed to bind the 3′ end of the poliovirus replicon. After fixing and overnight incubation in 70% ethanol, cells were washed twice in PBS; equilibrated in FISH Wash Buffer containing 2× SSC (Invitrogen 15557044) and 20% Formamide (Ambion AM9342) for 5 minutes at RT; and hybridized with Stellaris FISH probes labeled with Quasar 670 at 125 nM (Biosearch Technologies, Supplementary Table 1) overnight at 30° C. in Hybridization Buffer (containing 20% Formamide (Ambion AM9342), 2× SSC, 0.1 g/mL Dextran Sulfate (Fisher Sci BP1585-Dextran Sulfate), 1 mg/mL E. coli tRNA (Roche 10109541001), 2 mM Vanadyl ribonucleoside complex (NEB S1402S), and 0.1% TWEEN® 20 (VWR 97062-332) in nuclease free water). The next morning, the hybridization buffer was removed and cells were washed twice in FISH Wash Buffer; incubated in FISH Wash Buffer without probe for 30 minutes at 30° C.; washed three times with 2× SSC; counterstained with DAPI; and finally imaged in 2× SSC.

### Microscopy

Cells were imaged on a Nikon Ti2 with PFS4, a Nikon Motorized Encoded Stage, Lumencor SPECTRAX LIGHT ENGINE®, custom Semrock filters, and a Prime 95B sCMOS camera. Automated acquisition for snapshots and time-lapse was programmed in NIS Elements. The scope was equipped with an OKO stage top incubator with temperature-, humidity-, and CO₂-control, enabling long-term imaging. For imaging smFISH a 60×, objective was used. Otherwise, imaging was performed using a 10× or 20× ELWD objective.

### Survival Curve Analysis

Nude mice were engrafted with 5×10⁶ H446 cells on their hind flanks. Survival was 100% post engraftment and mice were distributed into groups based on tumor sizes to ensure comparable average tumor sizes between mice in each group. Mice were euthanized once tumors reached 2000 mm³.

### Imaging Analysis

Images were recorded and processed using NIS-Elements software, FIJI, MATLAB®, and Python Code. Time-lapse and smFISH image analysis were performed as described previously.

### Example 2

### Engineered S. typhimurium Autonomously Launches Viral RNA

To establish CAPPSID as a bacterial platform capable of delivering viral RNAs, S. typhimurium, a naturally facultative intracellular bacterium, was chosen. Upon uptake into the mammalian cell, these bacteria harboring genetically encoded environmental sensors triggered in situ transcription of viral RNA, along with bacterial and vacuolar lysis proteins to enable delivery of the viral genome into the host cytoplasm. S. typhimurium achieves invasion into host cells via macropinocytosis, and then survives within the Salmonella Containing Vacuole (SCV) by expressing a battery of genes encoded on Salmonella Pathogenicity Islands 1 and 2 (SPI-1 and SPI-2), respectively. S typhimurium leveraging SPI-1 and 2 associated genes, promoters, secretion systems, and localization have been previously engineered for the delivery of proteins and plasmids.

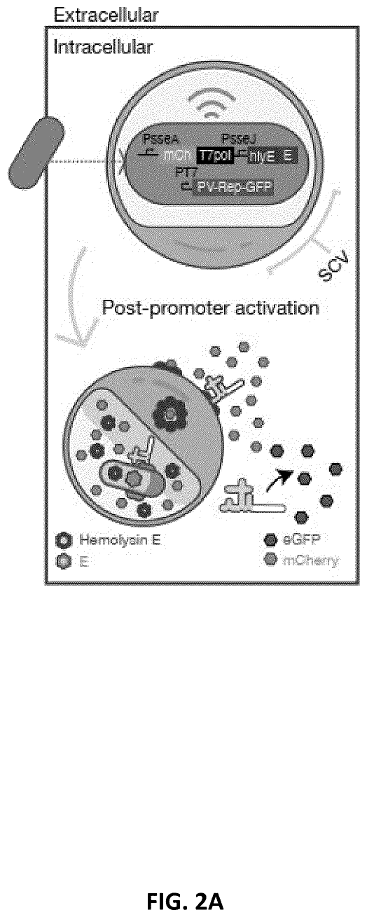

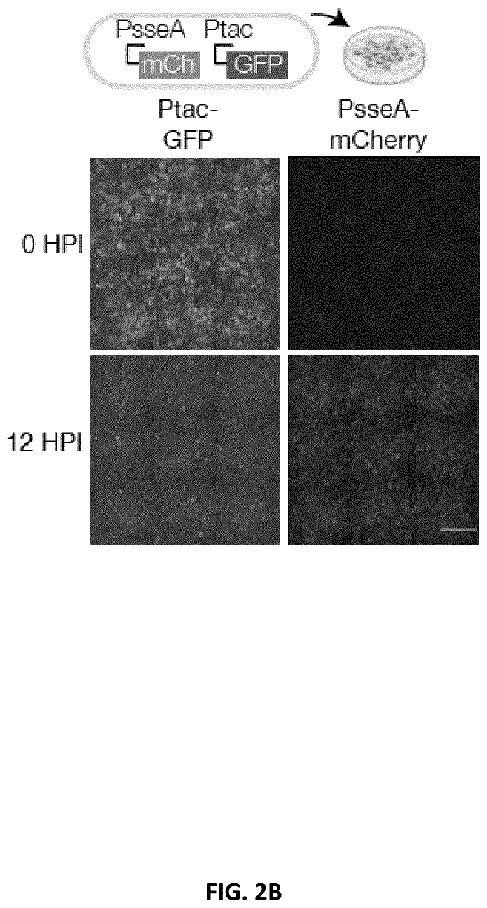

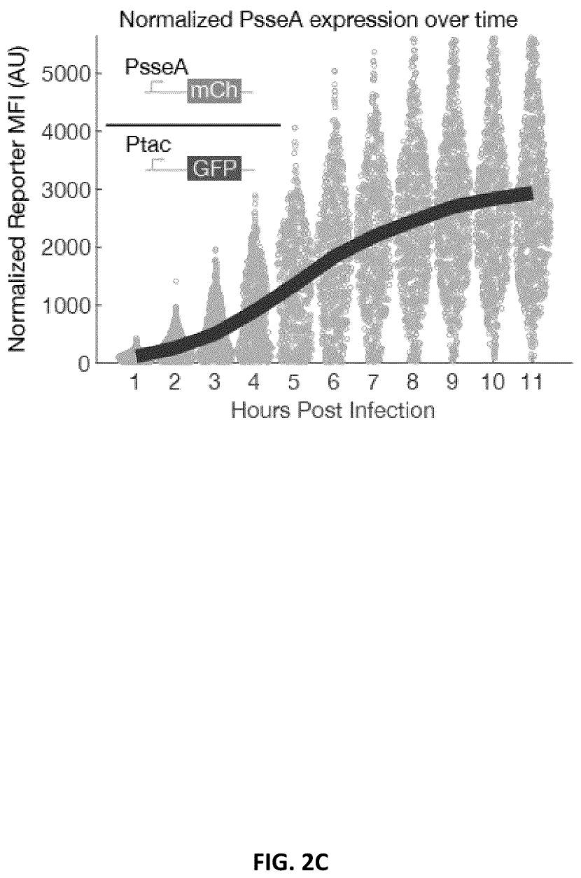

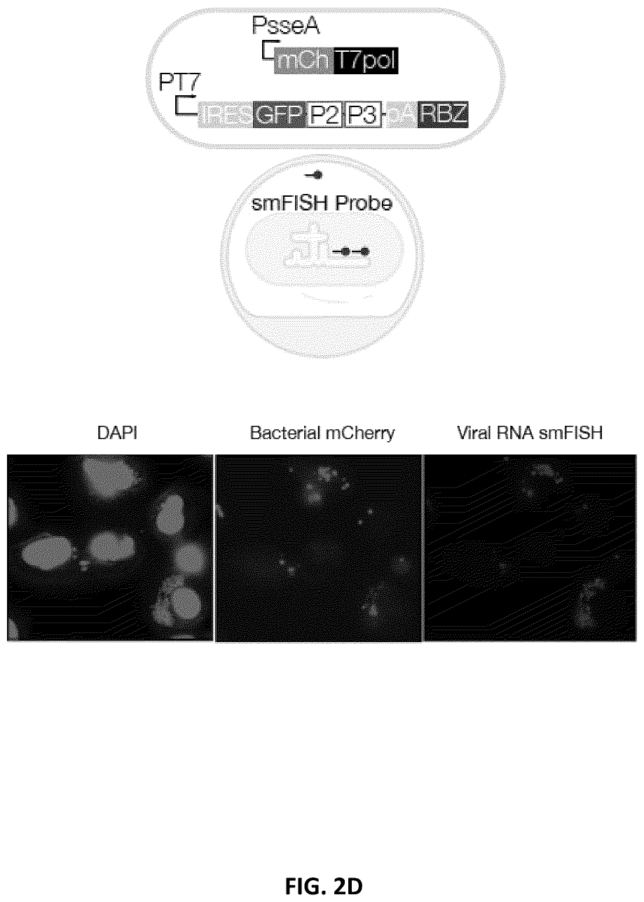

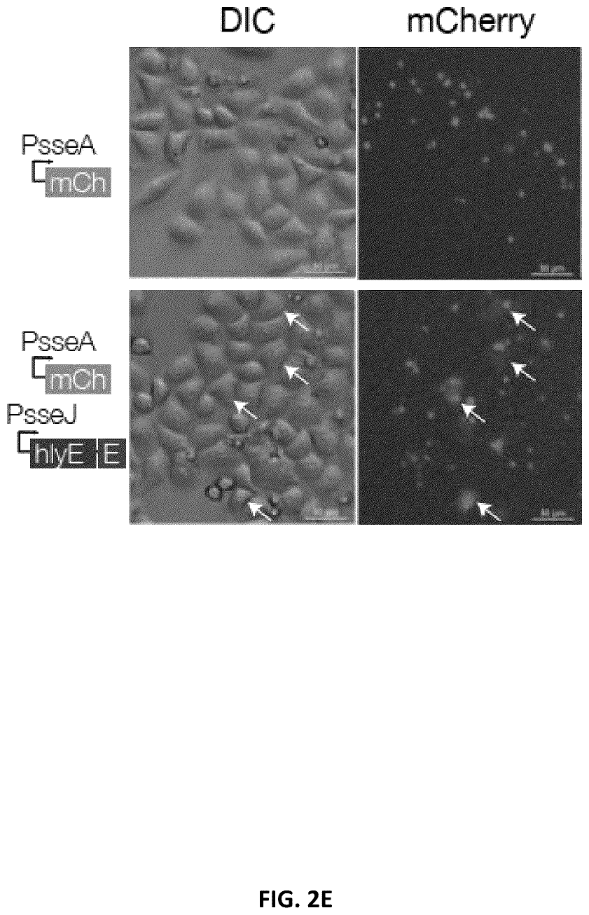

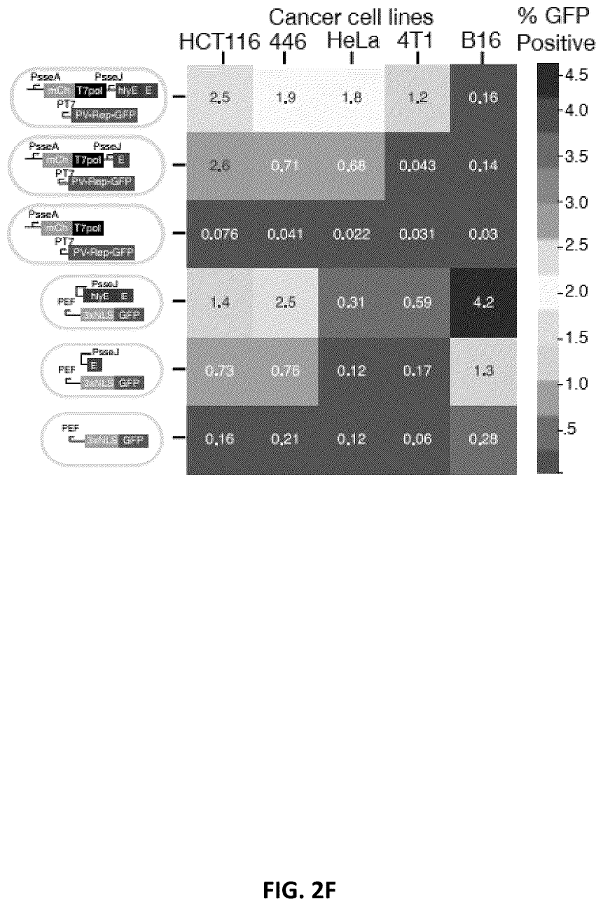

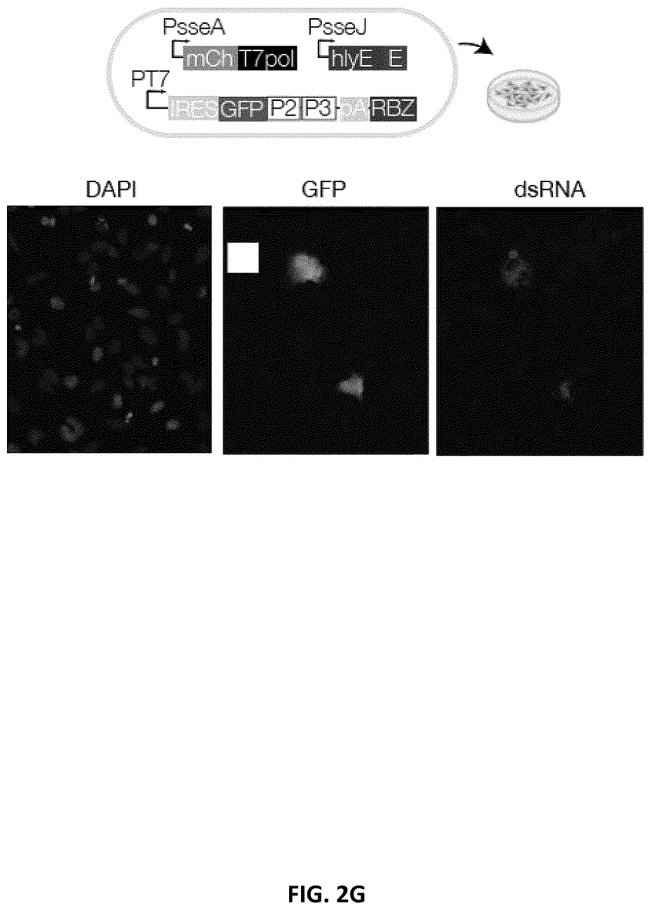

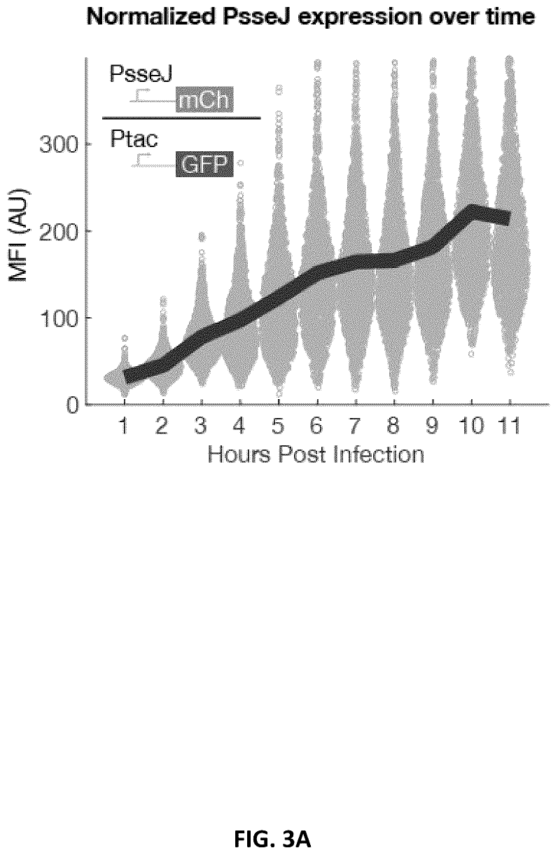

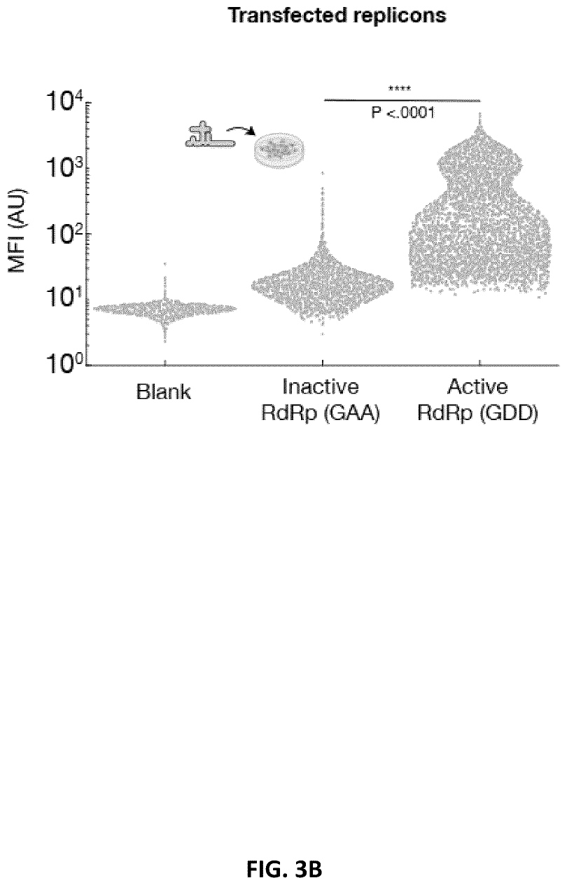

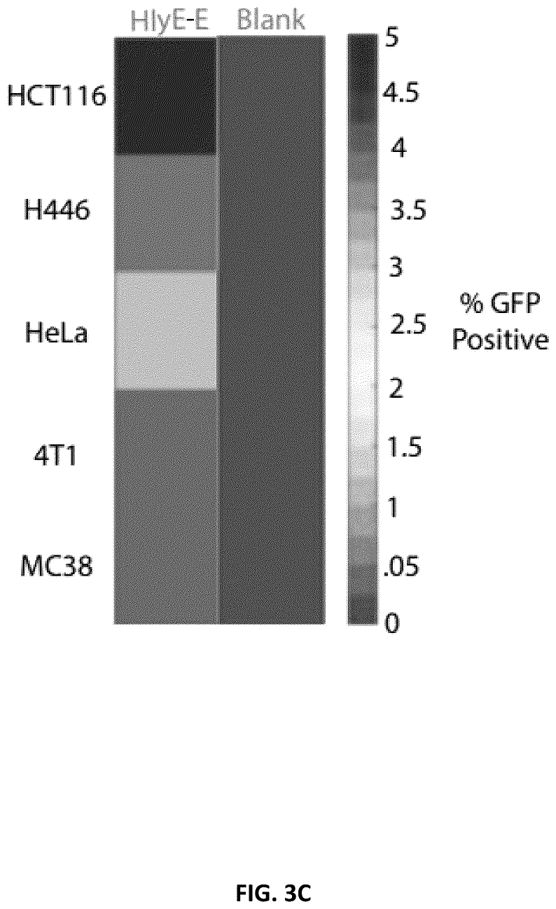

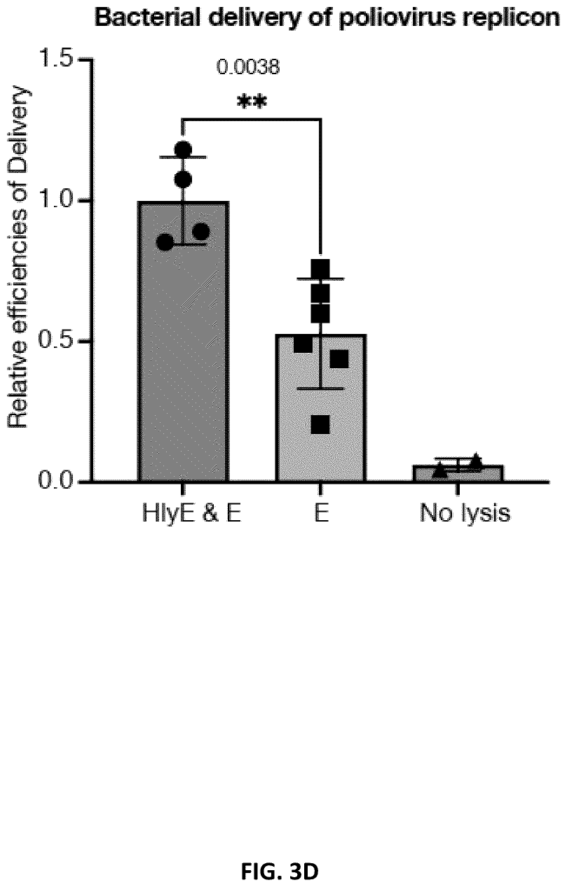

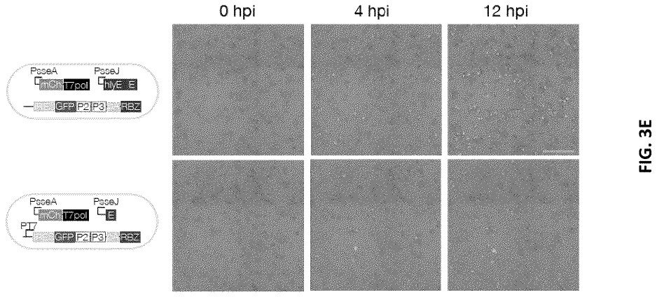

In order to exploit such a platform for viral RNA delivery, genes were chosen whose activity is restricted to the intravacuolar space and SPI-2 promoters were selected in order to drive viral RNA transcription in situ (FIG. 2A). SPI-2 promoters belonging to the sseA and sseJ genes, which are upregulated in the SCV, were used to trigger activation. Using time-lapse imaging, their temporal activity was evaluated within individual cancer cells in the attenuated S. typhimurium strain LH1301 (ΔaroA, ΔphoPQ) normalized to a constitutive GFP. Rapid activation of both promoters after entering mammalian cells was observed, with no signal present during the initial 30-minute inoculation of bacteria, suggesting that these promoters are tightly regulated and suitable for intracellular cargo expression (FIGS. 2B, 2C, and 3A).

Because prokaryotes do not incorporate 5′m7G caps required for typical mammalian translation, viral RNAs were chosen that rely instead on cap-independent translation. Compatible with this requirement, Picornaviridae recruit ribosomes through an elaborate secondary structure at the 5′ end of their genomes known as an internal ribosome entry site (IRES). To evaluate how such a platform might function across a range of lines, a poliovirus replicon was utilized where its structural proteins were replaced with the fluorescent reporter, GFP. This further serves to uncouple viral replication from spread. By transfection, active replication led to observing GFP levels 50- to 1000-fold higher than when the polymerase was mutationally inactivated, highlighting the utility of using self-amplifying RNA (FIG. 3B).

To couple transcription of this viral RNA to intracellular sensing, the PsseA promoter drives the strongly processive T7-RNA polymerase, which in turn transcribes the viral RNA off a complementary cDNA genome encoded on a plasmid. When this circuit is transformed into S. typhimurium LH1301 and used to invade Hela cells, these bacteria produce the full-length viral RNA, as measured by single-molecule fluorescence in situ hybridization (smFISH) using probes against the 3′ end of the 5.5 kb RNA (FIG. 2D).

Once transcribed, the viral genome must exit the bacterium and translocate through the SCV into the cytoplasm of the mammalian host in order to replicate. To optimize efficiency of this 2-step translocation, two distinct bacterial lytic proteins were used: Lysis protein E from phage (X174 that disrupts bacterial membranes, allowing the viral RNA to exit the lysed bacterium, and Hemolysin E (HlyE), which forms pores in the SCV, allowing the viral RNA to enter the host cytosol. These genes were expressed under the control of intracellular sensing promoter PsseJ, and complemented by a deletion of the sifA gene in S. typhimurium LH1301, whose loss further disrupts SCV integrity. When S. typhimurium carries this circuit into Hela cells, mCherry appeared to diffuse out of the SCV, filling the cytoplasm of the host cell, while in the absence of these lytic proteins, mCherry remained punctate, indicating restricted localization within vacuoles (FIG. 2E).

Finally, viral transcription and the lysis circuit were coupled together in order to evaluate whether the poliovirus replicon could be delivered into a range of cell types to launch replication. Strong GFP signals indicative of successful viral delivery were observed and replication in both mouse and human cell lines, including 4T1, B16, HCT116, HeLa, MC38, and H446 cells, were also observed, albeit with differing efficiencies (FIGS. 2F and 3C). Furthermore, active lysis (via E and HlyE) enabled dramatically more efficient delivery than in the absence of lytic proteins or when delivering plasmid-encoded GFP in most cell lines (FIGS. 3D and 3E). Finally, to confirm that this was not simply passive translation of the incoming genomic viral RNA, cells were stained with an antibody against long double-stranded RNA (dsRNA), a product of active viral replication, and strong signals were observed in cells that were also GFP positive (FIG. 2G). Together, these data show that lysing S. typhimurium is capable of successfully delivering actively replicating viral RNA.

### Example 3

### Delivery of Full-Length Oncolytic Virus by Engineered S. typhimurium Clears Subcutaneous SCLC Tumors

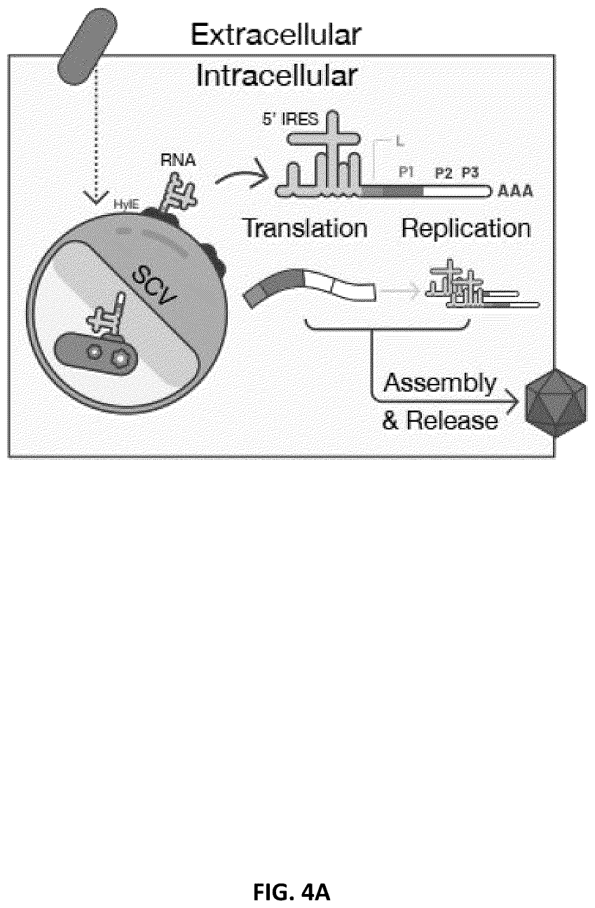

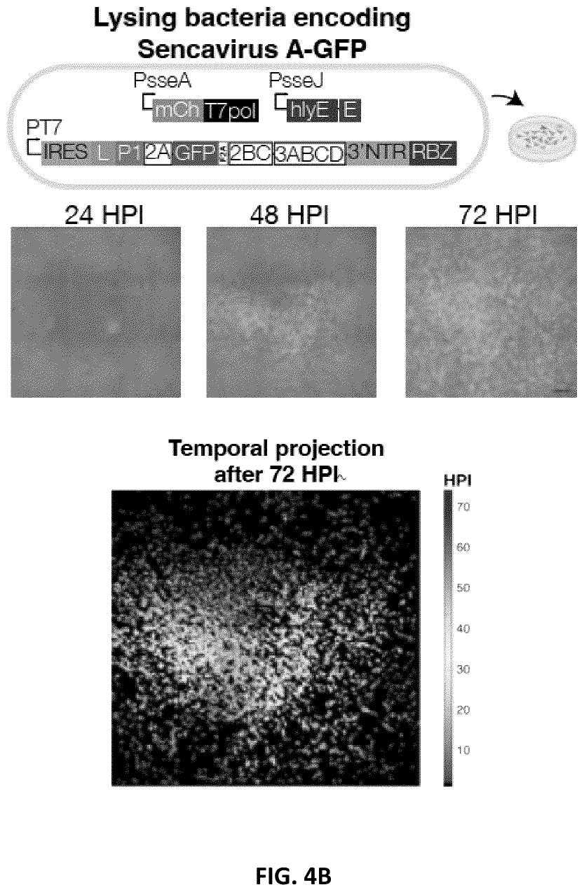

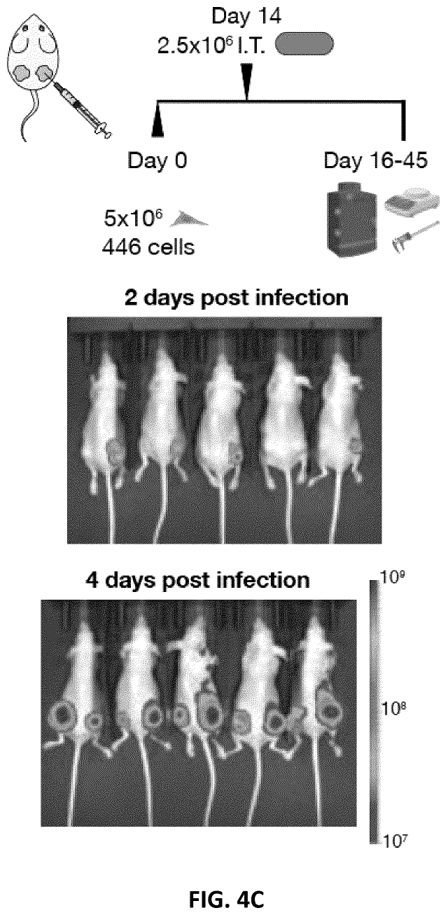

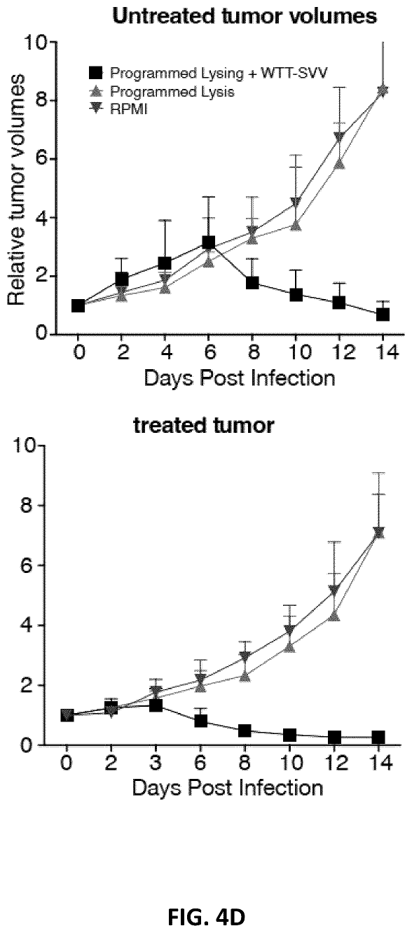

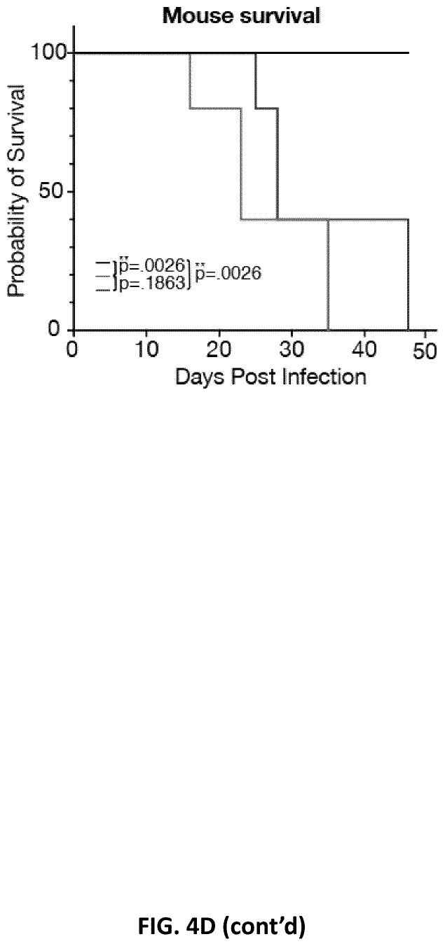

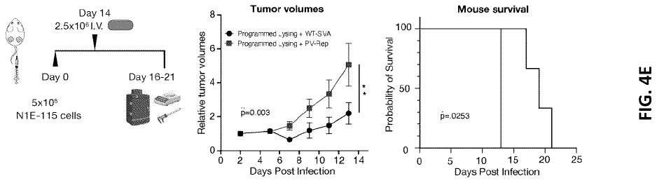

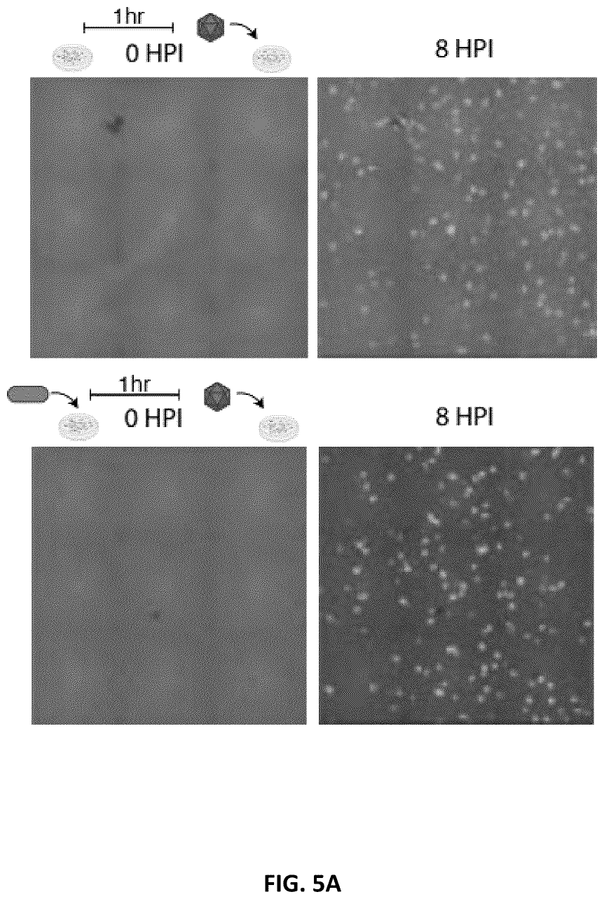

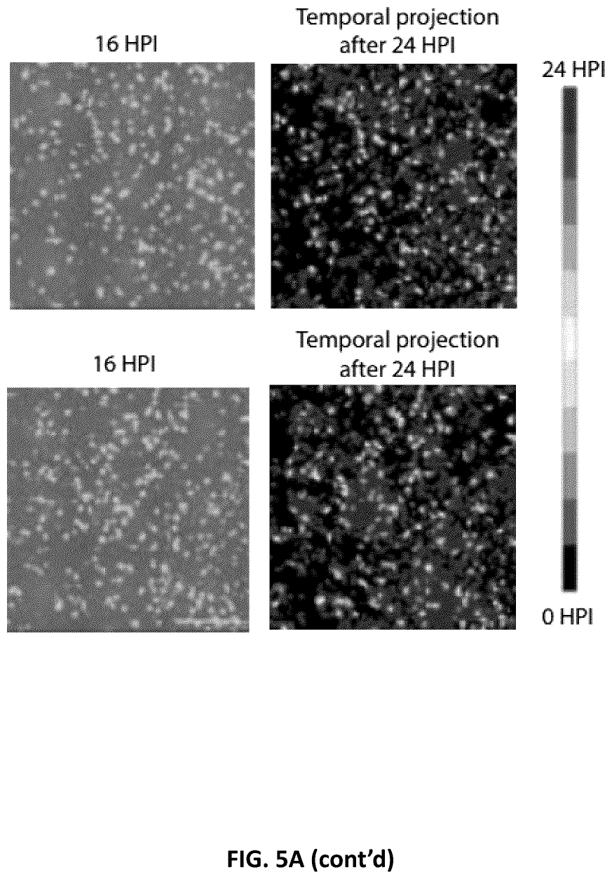

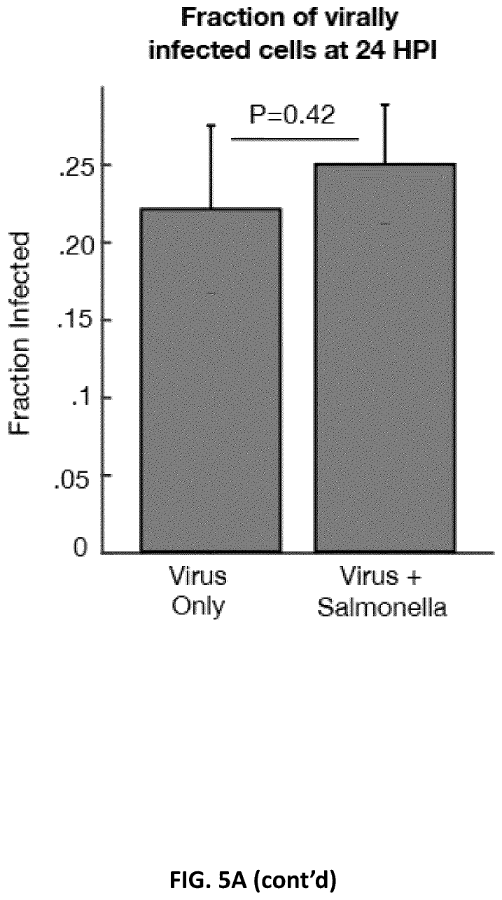

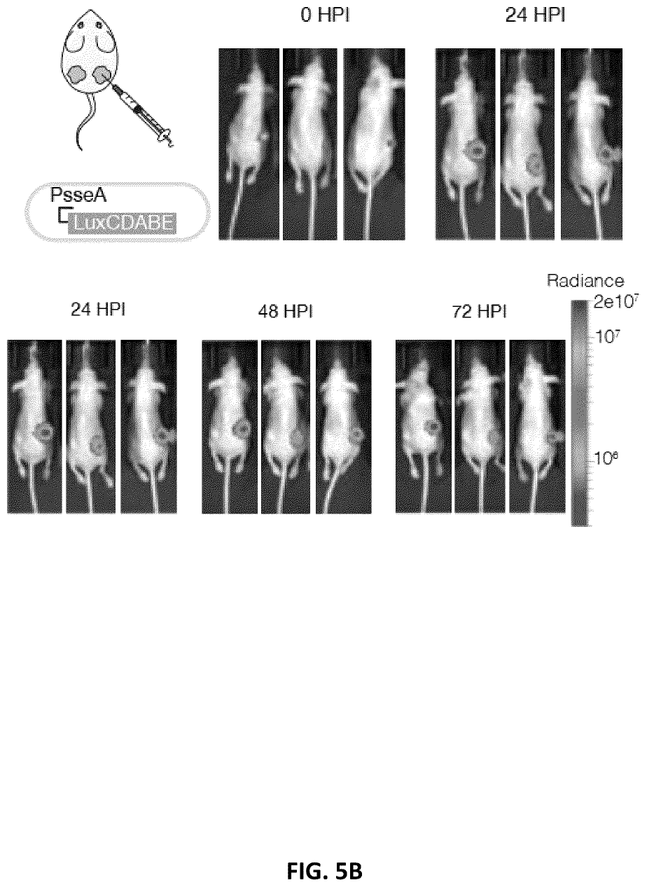

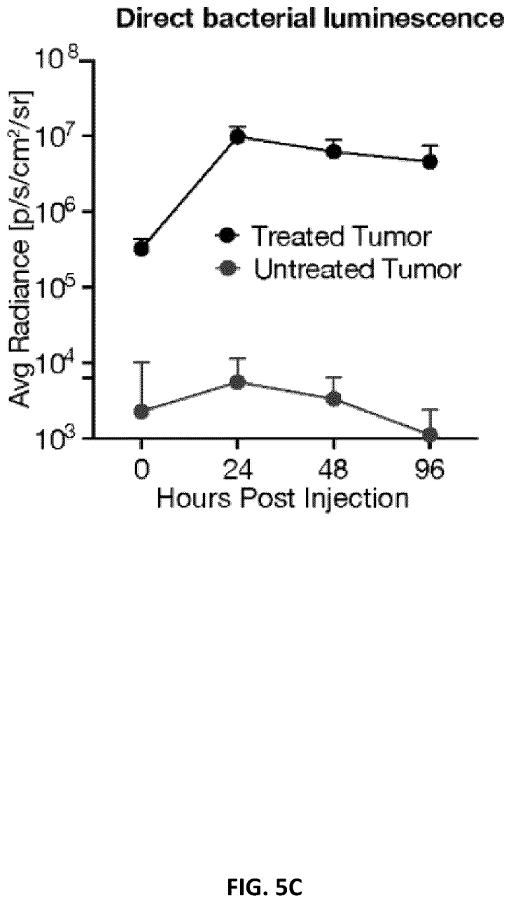

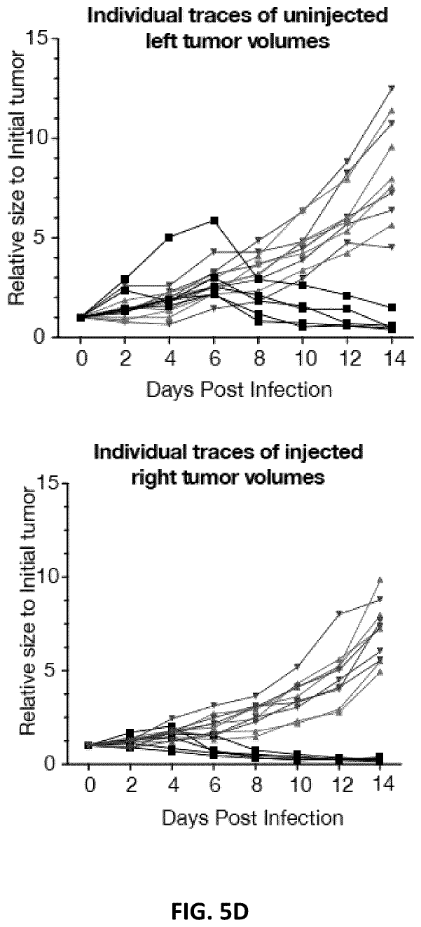

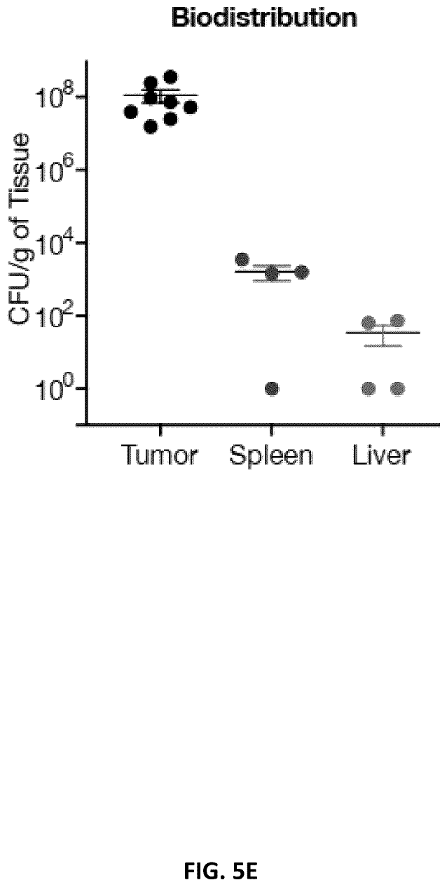

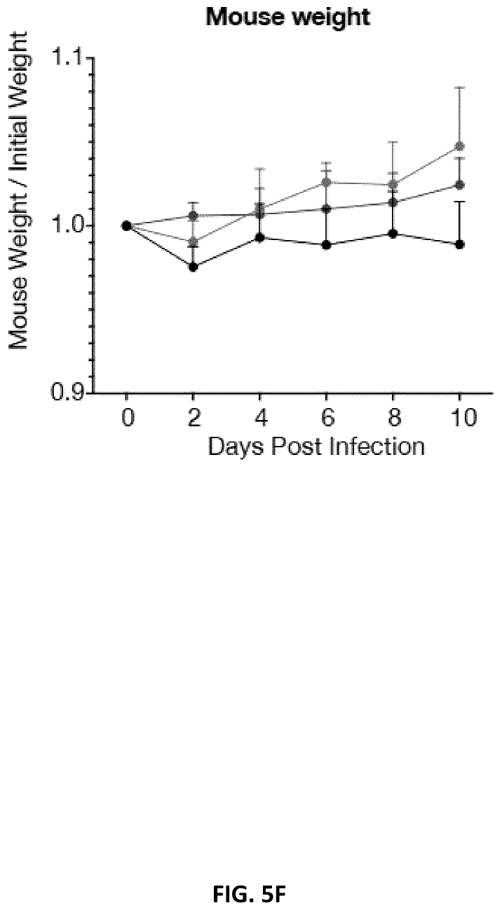

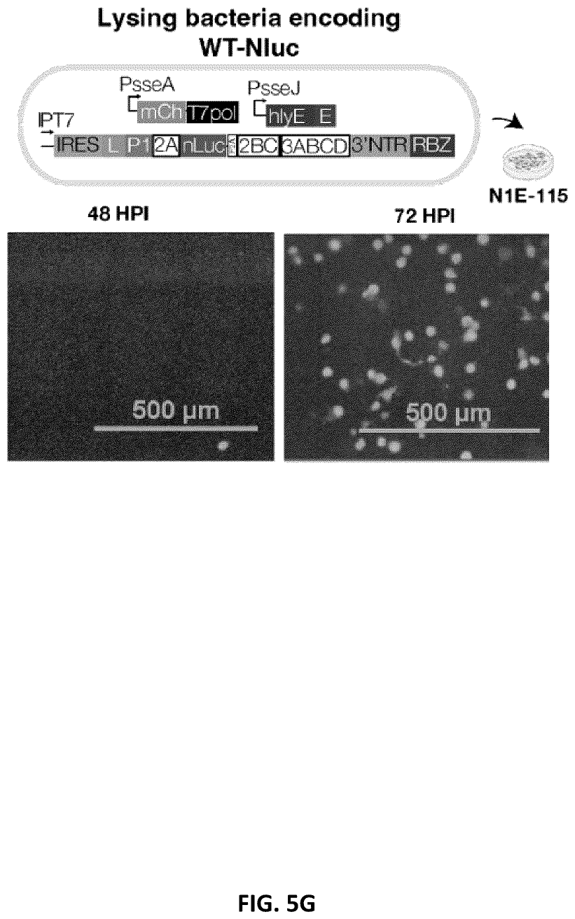

Because cells infected with S. typhimurium frequently die via induction of apoptosis and pyroptosis, a spreading virus could infect surrounding S. typhimurium-free cells, augmenting the overall therapeutic effect (FIG. 4A). For such a system to function, bacteria and virus must both be able to infect the target population, with the former not directly inhibiting the latter. Using Senecavirus A (SVA) reporter, SVA-GFP, viral spread was evaluated after being introduced one hour after bacterial pre-infection in H446 cells, a small cell lung cancer (SCLC) line. At 24 hours post infection there was no measurable reduction in the spread of virus in the presence or absence of bacteria (FIG. 5A).

In lysing S. typhimurium, the poliovirus replicon was replaced with SVA-GFP and inoculated into H446 cells to look for successful initial delivery events from bacteria and a subsequent capacity for spread throughout the same culture. The first wave of SVA-infected cells was observed at approximately 8 hours following bacteria inoculation, with viral spreading occurring continuously throughout the subsequent 60 hours (FIG. 3B). By 72 HPI, effectively all cells were SVA-positive, regardless of initial bacterial infection status These results demonstrate that even a low fraction of initially infected cells with S. typhimurium could deliver and launch the spread of an oncolytic virus across an entire monolayer of cells.

To determine whether CAPPSID carrying the full length SVA virus could achieve thi effect in vivo, nude mice were xenografted with bilateral hind flank H446 tumors. Then, right tumors were injected intratumorally (IT) with lysing S. typhimurium carrying SVA-NANOLUC® (a luminescent reporter) and imaged over time for luminescence. Two days post-infection, right-flank tumors showed luminescence so strong that it saturated the detector (FIG. 4C). Furthermore, at day four, the signal was additionally observable in left tumors that had not been injected with bacteria, suggesting productive viral infection in the right tumors and sufficient titer capable of viral translocation to left-flank tumors. In contrast, control bacteria recombinantly expressing their own luminescent protein, luxCDABE, under the control of PsseA, showed no detectable translocation to left tumors over the same time (FIGS. 5B and 5C).

Tumor volume measurements over more than 40 days showed complete regression of both left and right tumors in the treatment group within two weeks, whereas all tumors treated with only buffer or lysing bacteria alone continued to grow until reaching maximum allowable sizes (FIGS. 4D and 5D). The striking regression of tumors conferred 100% survival in mice treated with S. typhimurium carrying SVA-NANOLUC®, while all control mice treated with RPMI and lysing Salmonella alone succumbed to tumor burden. Mice experienced no decline in weight, nor detectable bacteria in the liver or spleen, despite appreciable loads present in the tumors, suggesting no adverse response to bacterial injections (FIGS. 5E and 5F). Taken together, CAPPSID with SVA-NANOLUC® is capable of launching a viral infection and clearing H446 tumors in vivo.

### Example 4

### Systemic Administration of CAPPSID Prolongs Survival of Immunocompetent Mice Bearing Syngeneic Neuroblastoma Tumors

After demonstrating efficacy of viral launch using IT injected bacteria in athymic mice unsusceptible to SVA, systemic administration in a fully immunocompetent model susceptible to the virus was examined. To achieve this, a syngeneic mouse model coupling the neuroblastoma cell line NIE-115 with A/J mice was utilized.

First, to demonstrate in vitro activity of the platform, the invasion assay was repeated and successful launch and spread of the virus by 72 HPI was observed (FIG. 5G). Next, mice were engrafted with double-hind flank NIE-115 tumors, and two weeks later bacteria were intratumorally injected. Following the mice over a course of one month, an initial weight loss in mice treated with bacteria was observed, but rebounded by 6 days post-injection and attenuated growth kinetics compared to both mock treatment and lysing bacteria alone, resulting in an improvement in overall survival (FIG. 5H).

Finally, the efficacy of systemic delivery of bacteria was evaluated in this syngeneic model when the bacterial vector was delivered systemically. Following intravenous injection of bacteria carrying either SVA-NANOLUCR or a non-spreading poliovirus replicon, a significant reduction in growth and an overall improved survival benefit with launch of the full-length Senecavirus A was observed (FIG. 4E). Together these data demonstrate the ability of this platform to attenuate tumor growth in a fully immunocompetent model when delivered either intratumorally or systemically.

### Example 5

### CAPPSID Enables Engineered Synergy Between Virus and Bacteria to Control Viral Spread

Improving efficacy and safety profiles of oncolytic viruses is achieved either at the cell surface level, where the receptor-binding domain is altered to more specifically recognize a target cell, or intracellularly, where replication of the virus is modulated positively or negatively by cell-type specific cytoplasmic or nuclear determinants. Having constructed a bacterial platform that successfully delivers both viral RNA as well as proteins into the host cytoplasm, further control viral replication was investigated.

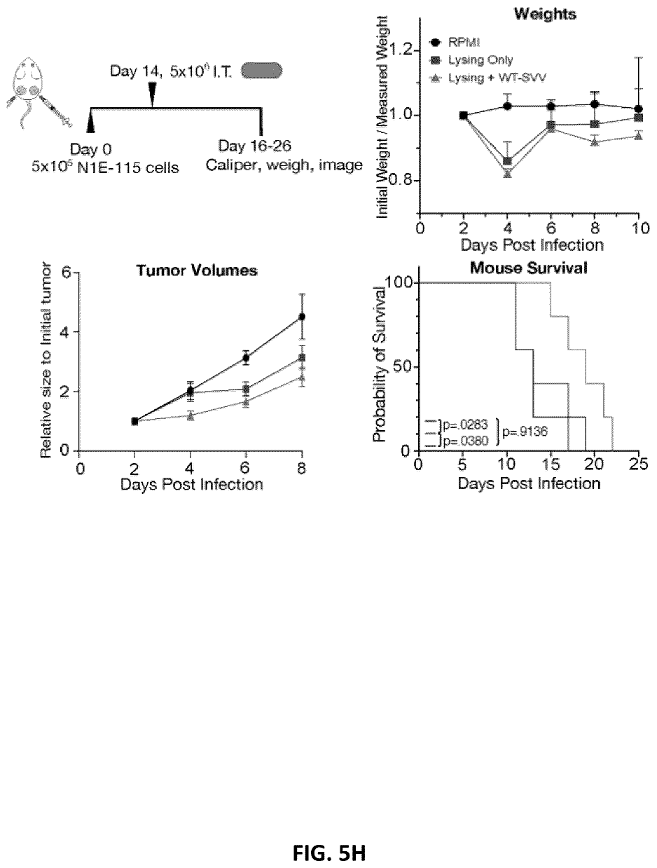

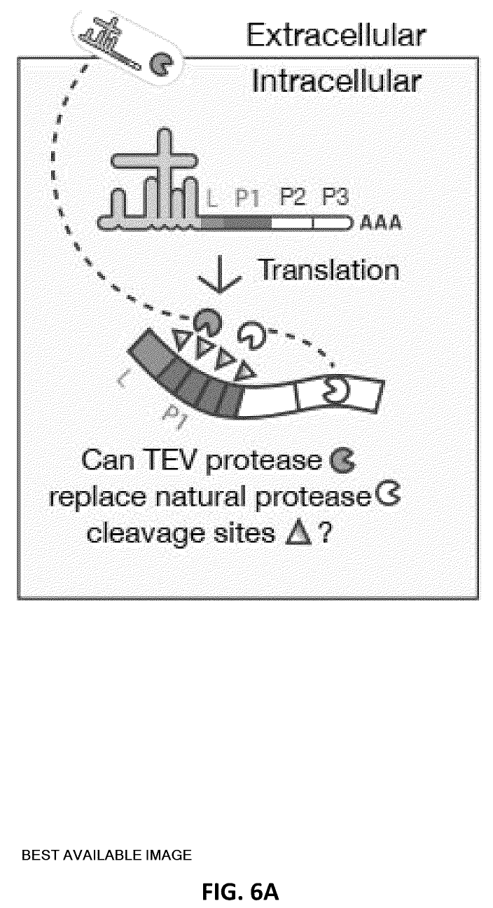

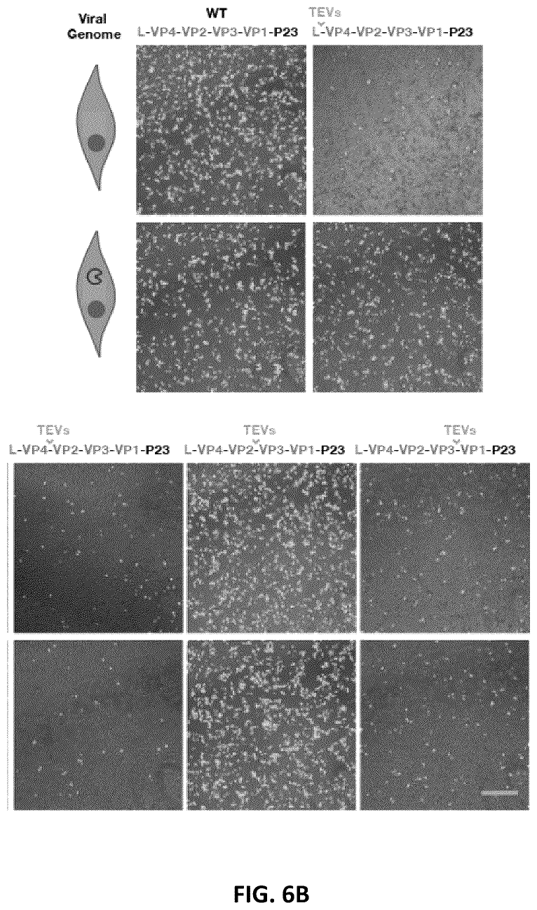

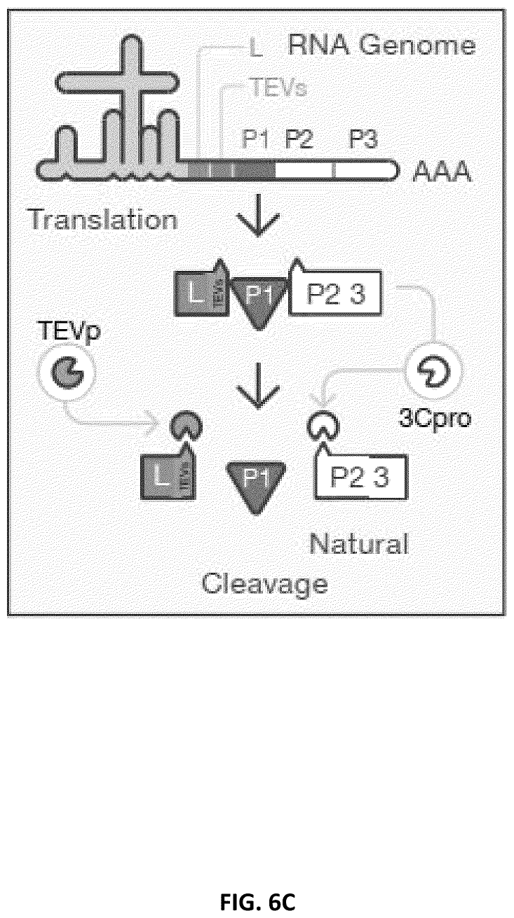

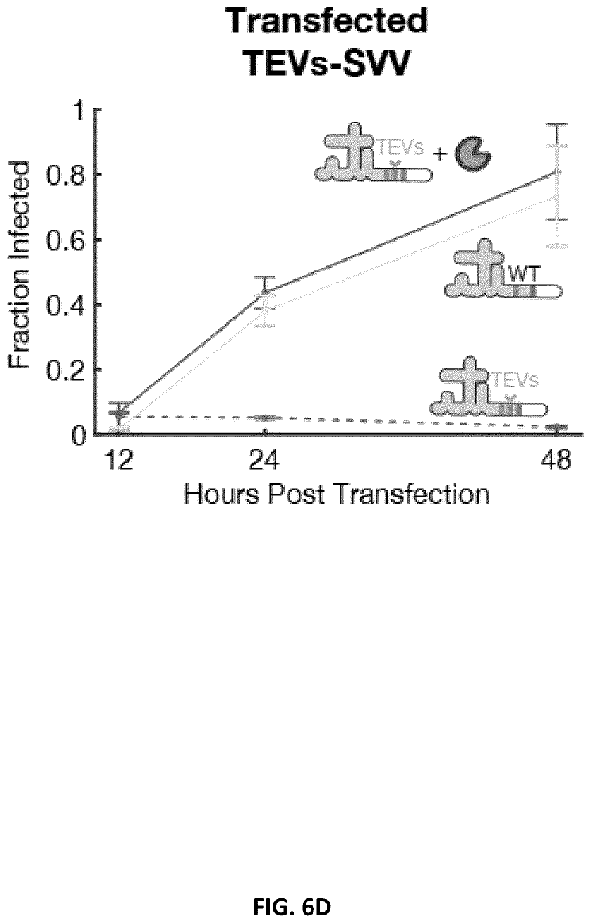

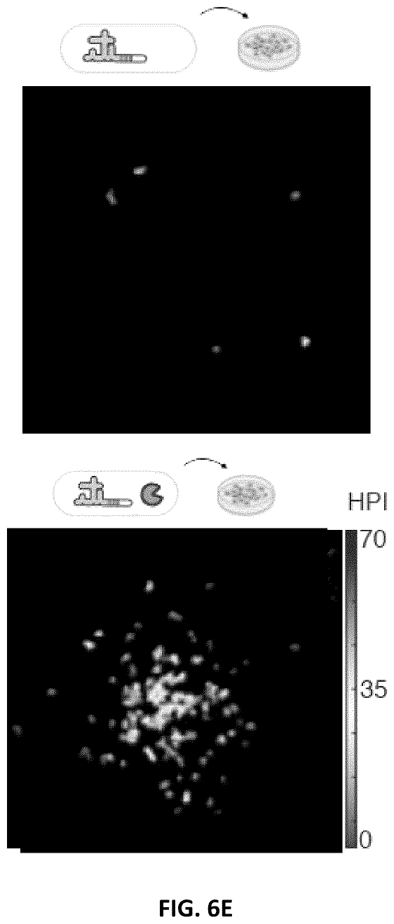

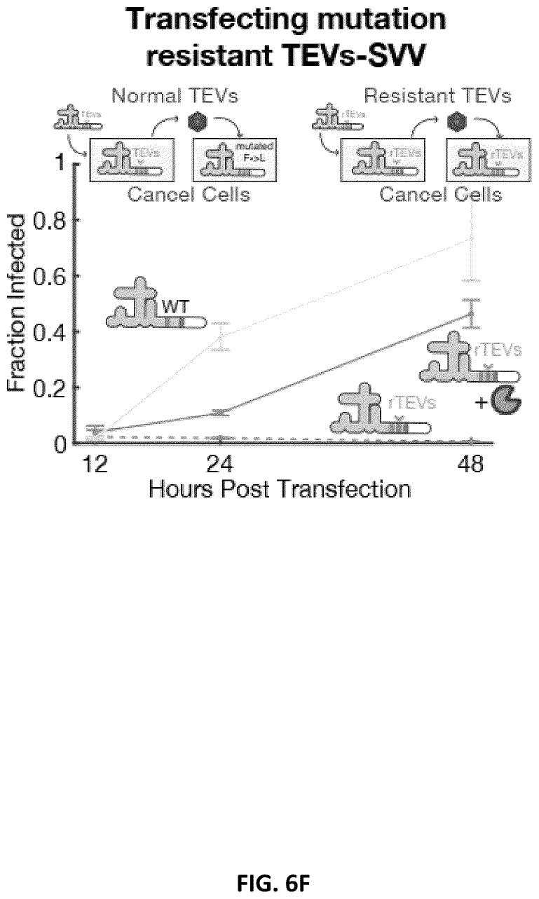

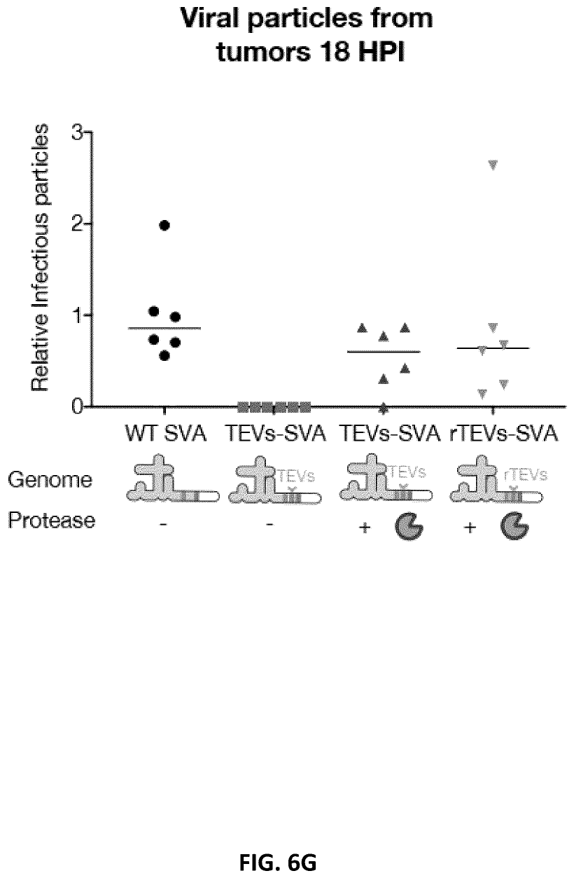

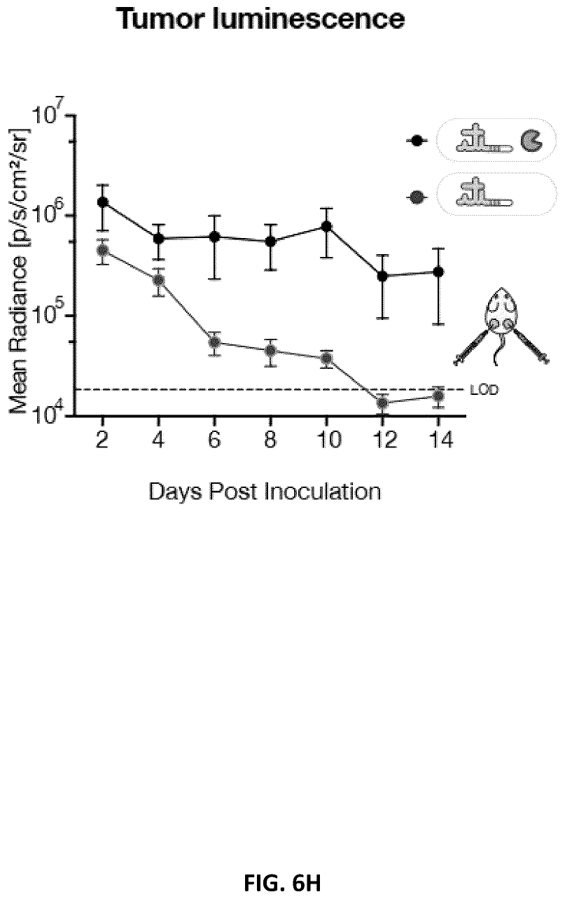

In the life cycle of picornaviruses, all proteins are translated first as one large open reading frame. This polyprotein must be then cleaved into individual constituents entirely by virally encoded proteases. Thus, shifting a cleavage event to an orthogonal protease expressed by bacteria would enable a control over the viral life cycle and such a protease might be delivered simultaneously by lysing bacteria (FIG. 6A). Due to its potential for recombinant expression and thorough characterization, Tobacco Etch Virus protease (TEVp) was chosen as the orthogonal protease. Furthermore, TEVp has the flexibility to recognize nearly all residues at the final position of the cognate TEV cleavage site (TEVs) sequence (ENLYFQ\hat{ }G) where cleavage occurs between the last two amino acids. This allows for the ability to retain the native N-terminal residue of the downstream protein following successful cleavage.

Investigating which natural cleavage site in SVA might be amenable to TEVs substitution, all four sites flanking each structural protein were screened. This would enable successful packaging of the virus in cells containing the protease, followed by viral entry and replication in surrounding cells. However, should the surrounding cell not contain TEVp, further spread would be restricted. Each of these four potential variants were transfected into either wild-type H446 cells or H446s constitutively expressing TEVp (FIG. 6B). When the TEVs were placed between the nonstructural Leader protein and the first structural protein VP4, the spreading of this variant became entirely dependent on TEVp and was capable of infecting surrounding cells at a rate equivalent to wild-type virus. Replacing the other natural cleavage sites with TEVs resulted in TEV-independent spread or abrogation of spread entirely ((FIGS. 6C and 6D).

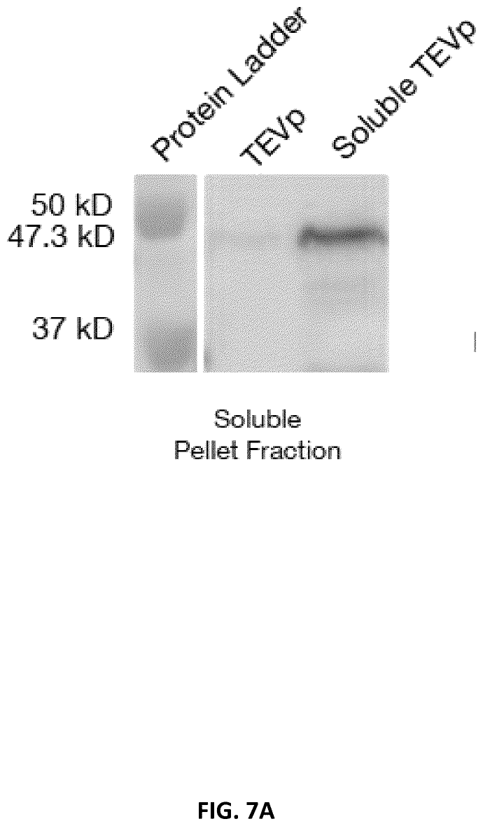

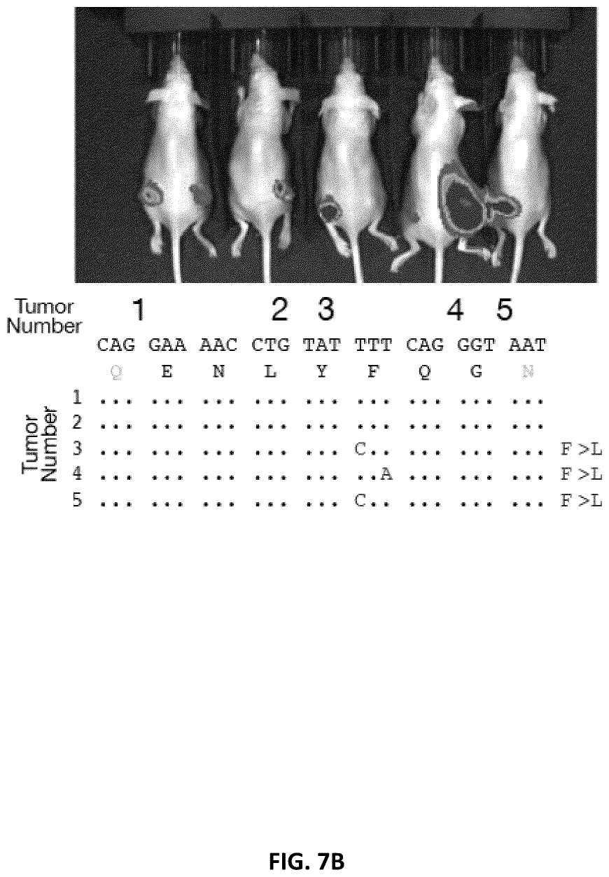

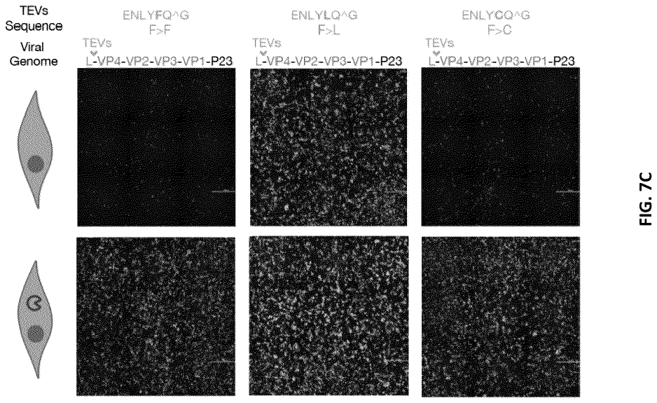

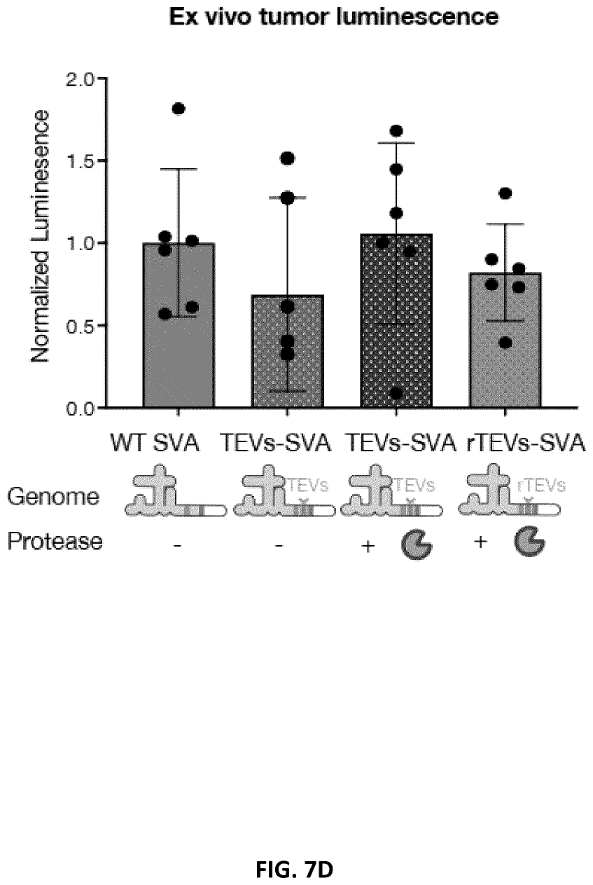

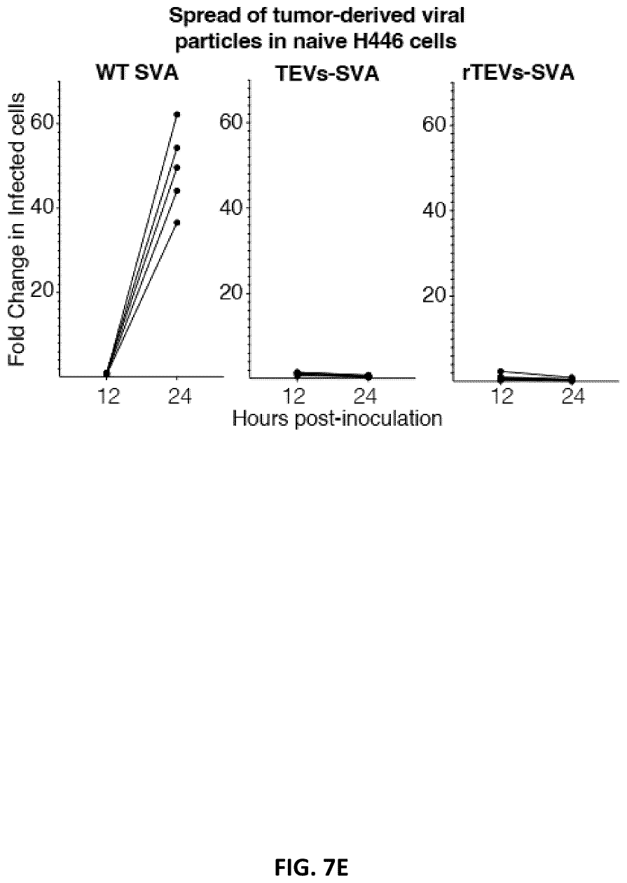

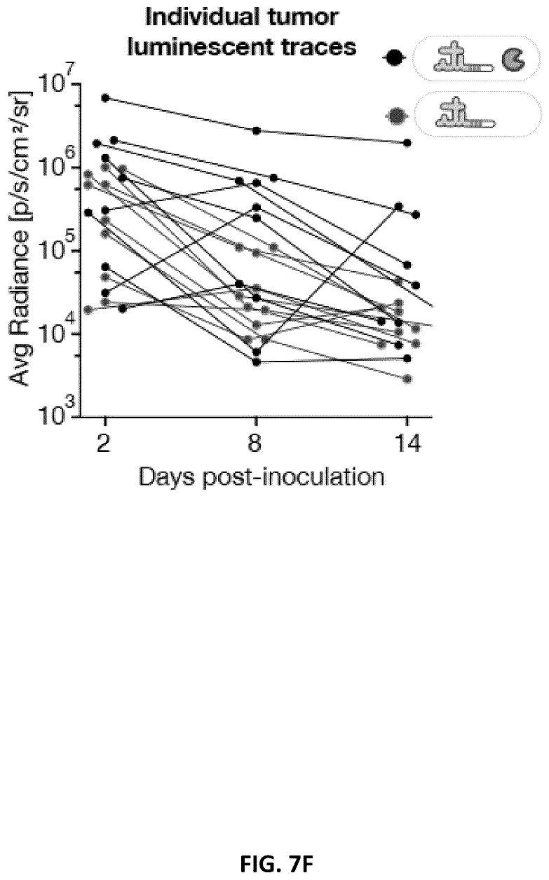

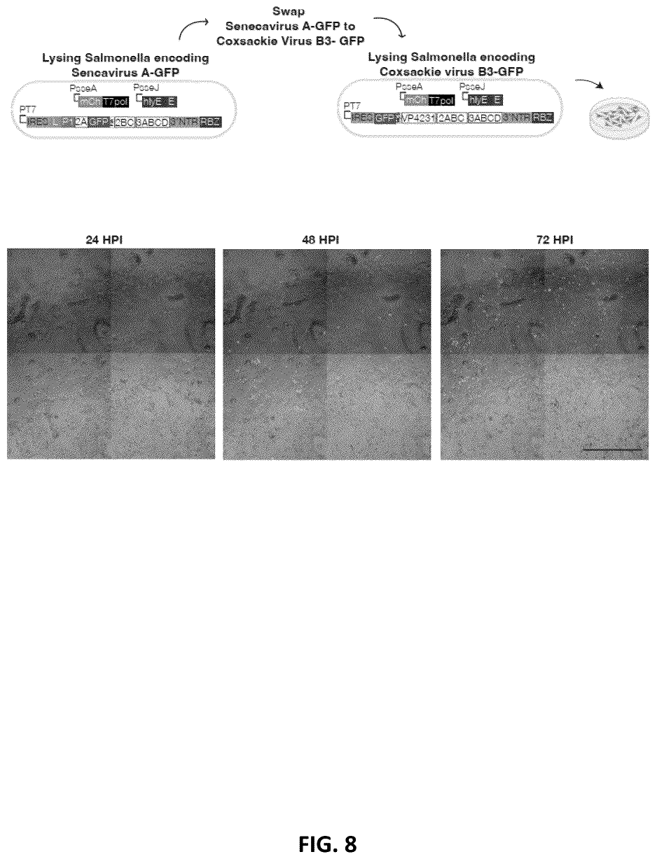

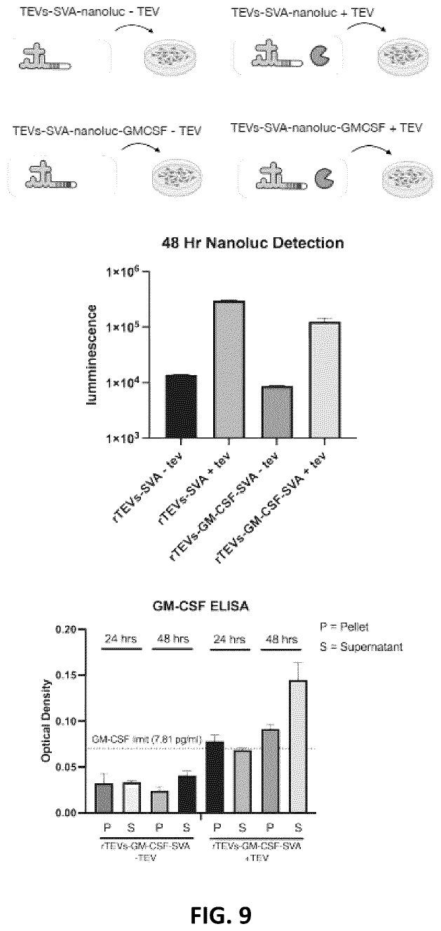

In order to couple the spread of this variant to co-infecting bacteria, lysing S. typhimurium were engineered to express TEVp under the control of a second PsseA intracellularly sensing promoter. Additionally, a series of mutations were incorporated into TEVp that were previously shown to improve the solubility of the protease (FIG. 7A). When the bacteria delivered a TEVp-dependent virus without bacterially produced TEVp, the virus launches but then fails to spread, as expected (FIG. 6E). However, when the S. typhimurium simultaneously delivered both the TEVp-dependent virus as well as TEVp, localized foci of spreading infection appeared (FIG. 6E).

This platform was next evaluated in vivo with the aim to characterize the stability of the engineered genome, owing to the high error rate of RNA-dependent RNA polymerases (RdRps) and potential to mutate away from TEVp-dependence. Tumors were injected IT with S. typhimurium delivering wild-type SVA-NANOLUC® and compared to TEVs-SVA-NANOLUC® with co-delivered protease. Over the course of one week, the luminescence of the group receiving bacterially delivered wild-type SVA-NANOLUC® continued to increase rapidly. In contrast, the signal from tumors injected with bacterially delivered TEV-dependent SVA remained lower than wild-type through day eight, but then began to increase (FIG. 7B). Sequencing viral RNA extracted from tumors that received the TEVs-SVA revealed that three out of five had a single-nucleotide polymorphism (SNP) in the TEVs, yielding two different ways of producing an identical phenylalanine-to-leucine (F->L) substitution (FIG. 7B). When this mutation was cloned into the viral genome and transfected directly into H446 with or without TEVp, this mutation was sufficient to achieve TEVp-independent spreading (FIG. 7C); a leucine at this site recapitulates the residue normally at this position immediately upstream of the scissile Q\hat{ }G cleavage site in the wild-type virus.

To prevent this “escape” mutation from occurring, an optimal TEVs sequence would be one where the codon for phenylalanine requires more than one SNP to revert into a leucine. While no codon like this for phenylalanine exists, previous interrogation of TEVs revealed that a cysteine at the phenylalanine site maintained TEVp-mediated cleavage, while being two SNPs away from reverting to a leucine. Indeed, an SVA variant with the modified TEVs sequence of ENLYCQ\hat{ }G only spread in the presence of TEVp, though at slightly reduced efficiency compared to the WT TEVs (FIGS. 6F and 7C). Thus a mutationally resistant variant of TEVp-dependent SVA (denoted rTEVs-SVA) was created.

Mice carrying double hind flank H446 tumors were injected IT with lysing S. typhimurium with and without TEVp, or WT-SVA alone. Twenty four hours following injection, tumors were harvested, homogenized, and assayed for luminescence ex vivo as a readout for replication originating from bacterial launch, as well as for viral titer measurements as an indication of successful packaging of the virus. The initial luminescence as measured ex vivo was statistically indistinguishable between groups, showing equivalent initial delivery from bacteria of WT virus compared to TEVp-dependent virus (FIG. 7D). Similarly, the number of viral particles produced by cells infected with TEVp-dependent virus was also statistically the same as WT virus delivery at launch (FIG. 6G). In contrast, no infectious particles were recovered when TEVp-dependent virus was delivered in the absence of TEVp, as expected (FIG. 6G). Furthermore, when naive cells in vitro were infected with tumor-harvested WT virus, spreading was observed, while tumors containing TEVp-dependent virus showed initial replication, but no further spread (FIG. 7E). Together, these data suggest that the initial launch and production of infectious viral particles are equally efficient between both WT and TEV-dependent viruses, and that engineered virus launched from bacteria was indeed TEVp-dependent.

Finally, when a cohort of mice injected with lysing S. typhimurium delivering TEVp-dependent virus with and without protease was measured longitudinally, luminescence from this mutation-resistant variant continued to remain present for up to two weeks following a single injection, while virus delivered without protease showed a complete loss of signal over the same time period. Over this time course, no increasing luminescent signals were observed, suggesting that reversion to TEVp-independence did not occur (FIGS. 6H and 7F).

While this invention has been disclosed with reference to particular embodiments, it is apparent that other embodiments and variations of the inventions disclosed herein can be devised by others skilled in the art without departing from the true spirit and scope thereof. The appended claims include all such embodiments and equivalent variations.

What is claimed is:

1. An engineered Salmonella typhimurium bacterium, comprising:
(a) a lysis circuit comprising a first heterologous nucleic acid encoding a first polypeptide that causes bacterial lysis and a second heterologous nucleic acid encoding a second polypeptide that causes vacuolar lysis; and
(b) a third heterologous nucleic acid encoding a virus.

2. The bacterium of claim 1, wherein the first polypeptide is lysis protein E from phage qX174 and the second polypeptide is Hemolysin E.

3. The bacterium of claim 1, wherein the third heterologous nucleic acid further comprises an intracellular sensing promoter.

4. The bacterium of claim 1, wherein the virus is a picornavirus.

5. The bacterium of claim 4, wherein picornavirus is Seneca A virus.

6. The bacterium of claim 1, further comprising a fourth heterologous nucleic acid encoding an orthogonal protease.

7. The bacterium of claim 6, wherein the fourth heterologous nucleic acid further comprises an intracellular sensing promoter.

8. The bacterium of claim 6, wherein the orthogonal protease is Tobacco Etch Virus protease.

9. The bacterium of claim 6, wherein the virus comprises one or more orthogonal cleavage sites.

10. The bacterium of claim 9, wherein the third heterologous nucleic acid encoding the virus comprises one or more optimized codons encoding the one or more orthogonal cleavage sites.

11. A method of treating a tumor in a subject comprising administering a therapeutically effective amount of engineered Salmonella typhimurium bacteria, wherein the bacteria comprise:
(a) a lysis circuit comprising a first heterologous nucleic acid encoding a first polypeptide that causes bacterial lysis and a second heterologous nucleic acid encoding a second polypeptide that causes vacuolar lysis; and
(b) a third heterologous nucleic acid encoding a virus.

12. The method of claim 11, wherein the first polypeptide is lysis protein E from phage qX174 and the second polypeptide is Hemolysin E.

13. The method of claim 11, wherein the third heterologous nucleic acid further comprises an intracellular sensing promoter.

14. The method of claim 11, wherein the virus is a picornavirus.

15. The method of claim 14, wherein picornavirus is Seneca A virus.

16. The method of claim 11, further comprising a fourth heterologous nucleic acid encoding an orthogonal protease.

17. The method of claim 16, wherein the fourth heterologous nucleic acid further comprises an intracellular sensing promoter.

18. The method of claim 16, wherein the orthogonal protease is Tobacco Etch Virus protease.

19. The method of claim 16, wherein the virus comprises one or more orthogonal cleavage sites.

20. The method of claim 19, wherein the third heterologous nucleic acid encoding the virus comprises one or more optimized codons encoding the one or more orthogonal cleavage sites.
